(19) United States
(12) Patent Application Publication
(10) Pub. No.: US 2012/0208254 A1
(43) Pub. Date: Aug. 16, 2012

(54) BIOREFINERY SYSTEM, COMPONENTS THEREFOR, METHODS OF USE, AND PRODUCTS DERIVED THEREFROM

(75) Inventors: Michael Francis Smith, Whitefish, MT (US); James Michael Rockwell, JR., Whitefish, MT (US)

(73) Assignee: Algae Aqua-Culture Technology, Inc., Whitefish, MT (US)

(21) Appl. No.: 13/354,281

(22) Filed: Jan. 19, 2012

Related U.S. Application Data
(60) Provisional application No. 61/434,353, filed on Jan. 19, 2011.

(51) Int. Cl. C12M 1/42; C12P 1/00; C12P 3/00; C12M 1/36; C12P 5/02

(52) U.S. Cl. 435/167; 435/292.1; 435/286.1; 435/168; 435/41

(57) ABSTRACT

Embodiments of the present disclosure provide systems, components, methods directed to generating energy and output products from biomass in a biorefinery system. The systems, components, and methods can be used alone or in combination as part of an integrated biorefinery system.

### CROSS-REFERENCE TO RELATED APPLICATION

This application claims the benefit of Provisional Application No. 61/434353, filed Jan. 19, 2011, the disclosure of which is hereby expressly incorporated in its entirety by reference herein.

### BACKGROUND

Expanding industrialization and increasing populations around the world continues to create an ever-increasing demand for energy, food, and potable water, while at the same time increasing the production of waste and potentially climate-altering greenhouse gases. It is well documented in the art that historical dependence on fossil fuels is becoming less reliable and/or more costly to manage its waste by-products. Similarly, conventional large-scale agriculture practices and the increasing presence of industrial waste run-off has reduced soil nutrient levels and negatively impacted natural and man-made water supplies, all of which reduce our ability to produce sustainable, nutritious food supplies for our communities.

Accordingly, the need and effort to identify and create means for generating alternative sources for renewable energy, as well as means for sequestering greenhouse gases, increasing soil viability, and remediating water supplies is well documented in the art.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a biorefinery system is provided. The system generally includes a photobioreactor system, an anaerobic bioreactor system, and an enclosure for containing at least a portion of the photobioreactor system and at least a portion of the anaerobic bioreactor system, wherein the enclosure has an environment for growing plant life.

In accordance with another embodiment of the present disclosure, a method of growing plant life in a greenhouse system is provided. The method generally includes forming an enclosure, wherein at least a portion of the enclosure is configured for receiving solar energy; disposing at least a portion of a photobioreactor system in the enclosure; and disposing at least a portion of an anaerobic bioreactor system in the enclosure.

In accordance with another embodiment of the present disclosure, a photobioreactor system for growing an algal colony is provided. The system generally includes a source of exhaust gas; a raceway system including a plurality of raceways configured to consume the exhaust gas to grow an algal colony; and a valve system for draining the algal colony from at least one of the raceways, wherein each of the plurality of raceways is positioned to be adjacent the valve system.

In accordance with another embodiment of the present disclosure, a method of growing an algal colony is provided. The method generally includes providing a photobioreactor system having a raceway system including a plurality of raceways; delivering exhaust gas to the algal colony; and after the algal colony reaches a predetermined colony density, draining the algal colony using a valve system, wherein each of the plurality of raceways is positioned to be adjacent the valve system.

In accordance with another embodiment of the present disclosure, a biorefinery system for sequestering exhaust gases to produce energy is provided. The system generally includes a biomass pyrolysis device configured to consume cellulosic biomass to produce exhaust gases; and a photobioreactor system configured to consume the exhaust gases from the biomass pyrolysis device to grow an algal colony.

In accordance with another embodiment of the present disclosure, a method of sequestering carbon dioxide is provided. The method generally includes obtaining carbon dioxide from a biomass pyrolysis system; and directing the carbon dioxide to an algal colony for consumption.

In accordance with another embodiment of the present disclosure, a soil regeneration product is provided. The product generally includes a carbon to nitrogen ratio in the range of about 2:1 to about 40:1; and a potassium content in the range of about 0.5 to about 7.0 percent.

In accordance with another embodiment of the present disclosure, a soil regeneration product is provided. The product generally includes a carbon to nitrogen ratio in the range of about 2:1 to about 40:1; and a second component selected from the group consisting of a potassium content in the range of about 0.5 to about 7.0 percent,
a sulfate content in the range of about 0.15 to about 1.3 percent,
a calcium content in the range of about 0.5 to about 6.8 percent,
a manganese content in the range of about 100 to about 350 mg/L,
a nitrogen content in the range of about 0.4 to about 2.0 percent,
a phosphorous content in the range of about 0.4 to about 1.5 percent,
a sodium content in the range of about 0.5 to about 18 percent,
a zinc content in the range of about 84 to about 233.1 mg/L,
an iron content in the range of about 600 to about 2500 mg/L,
a boron content in the range of about 5 to about 150 mg/L, and combinations thereof.

In accordance with another embodiment of the present disclosure, a method of remediating water is provided. The method generally includes generating a organic carbon product using a biomass pyrolysis device; and filtering water containing a first level impurities using the organic carbon product to produce water containing a second level of impurities, wherein the second level of impurities is less than the first level of impurities.

In accordance with another embodiment of the present disclosure, a control system for a biorefinery system is provided. The control system generally includes a biological process; and a plurality of autonomous agents for controlling a plurality of components in the biorefinery system, wherein one of the plurality of autonomous agents is a governing agent.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic of a biorefinery system, including a photobioreactor system, an anaerobic reactor system, a biomass pyrolysis system, and an energy conversion system in accordance with one embodiment of the present disclosure;

FIG. 2-4 are views of various embodiments of raceways for a photobioreactor system in accordance with embodiments of the present disclosure;

FIG. 5 is a top view of a multi-raceway photobioreactor system in accordance with one embodiment of the present disclosure;

FIGS. 6A and 6B are perspective views of a selector valve used in the multi-raceway photobioreactor system of FIG. 5;

FIG. 7 is a side cross-section view of the multi-raceway photobioreactor system of FIG. 5;

FIGS. 8A and 8B are respective top and side views of an alternate embodiment of a selector valve and water return system for use in a multi-raceway photobioreactor system, for example, of FIG. 5;

FIG. 9 is a process flow diagram for the biomass conversion process in an anaerobic bioreactor system in accordance with one embodiment of the present disclosure;

FIG. 10 is a schematic for a anaerobic bioreactor system in accordance with one embodiment of the present disclosure;

FIG. 11A is a schematic of a greenhouse system in accordance with one embodiment of the present disclosure;

FIG. 11B is a perspective view of an exemplary greenhouse system in accordance with one embodiment of the present disclosure;

FIG. 12 is a side cross-sectional view of a biomass pyrolysis system in accordance with one embodiment of the present disclosure;

FIG. 13 is a side view of a biomass loading system for a multi-biomass pyrolysis system;

FIG. 14 is a schematic of a biorefinery system, including a photobioreactor system, an anaerobic reactor system, a thermal energy source, and an energy conversion system in accordance with another embodiment of the present disclosure; and

FIG. 15-19 are schematics of various control systems for biorefinery systems in accordance with embodiments of the present disclosure.

### DETAILED DESCRIPTION

Embodiments of the present disclosure provide systems, components, and methods directed to generating energy and output products from biomass in a substantially closed loop system. The systems, components, and methods can be used alone or in combination as part of an integrated biorefinery system.

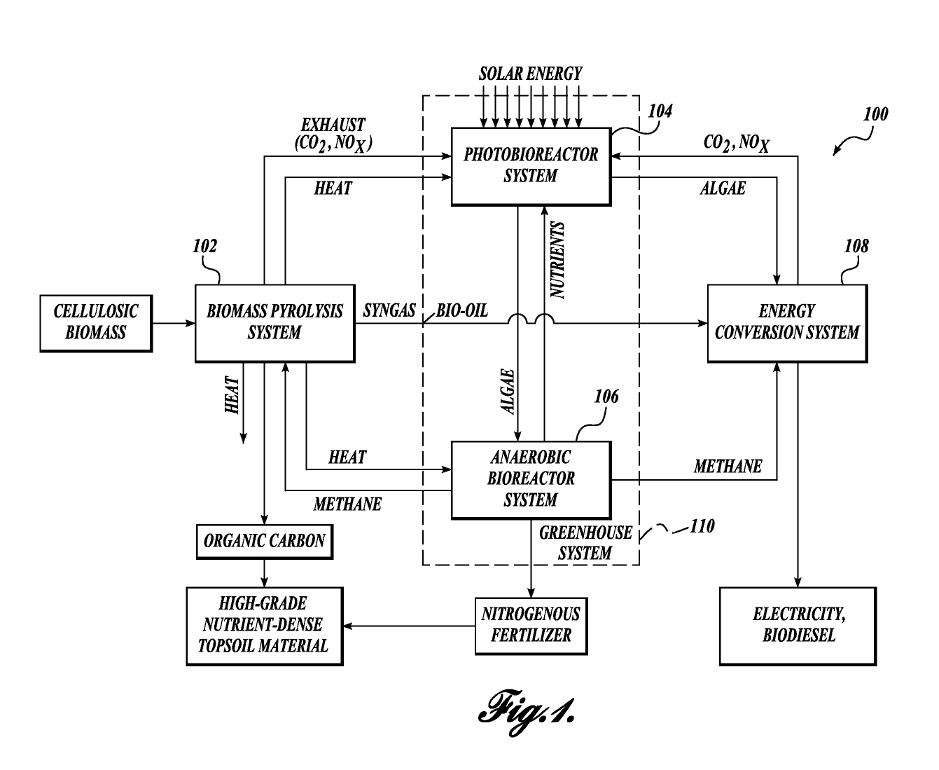

Referring to FIG. 1, one embodiment of component interrelationship in a biorefinery system 100 in accordance with the present disclosure is shown. The biorefinery system 100 generally includes a biomass pyrolysis system 102, a photosynthetic bioreactor system 104, and an anaerobic bioreactor system 106. The biorefinery system 100 may further include an energy conversion system 108, for example, for converting methane to electricity.

An optional greenhouse 110 may be configured contain one or more of the components in the system 100 and provide an environment to grow plant life. For example, in the illustrated embodiment, the greenhouse 110 is designed to contain the photosynthetic bioreactor system 104 and the anaerobic bioreactor system 106. Although shown as a complete system 100 in FIG. 1, it should be appreciated that embodiments of the present disclosure may be directed to one or more individual components shown in the system 100.

Biorefinery systems of the present disclosure, for example, as seen in FIG. 1, and their components may be used in a wide range of industries and applications, for example, anywhere it is desired to manage natural or man-made biomass or biomass waste, including woody biomass waste. In that regard, one input into the system is biomass, particularly woody biomass, including wood waste and hog fuel, macadamia nut shells, weeds, stover, and the like. Non-limiting examples of suitable industries and applications producing such biomass may include ranches, farms, and other agricultural applications including, for example, macadamia nut farms; local communities that produce yard and/or food waste; lumber mills, paper mills, and other wood-processing industries; industries and communities in tropical climates where management of naturally-occurring biomass is an issue, and the like.

Outputs from the system may include soil regenerating products, such as fertilizers, soil amendments, and soil regenerating products. Therefore, in accordance with embodiments of the present disclosure, useful industries and applications include communities and industries desiring access to high-grade, nutrient-dense, organic soil regenerating products. Therefore, embodiments of the present disclosure also feature compositions, methods, and means for generating soil regenerating products useful for organic plant cultivation and other agricultural applications.

The biorefinery system described herein is competent to act as a biomimetic system, emulating the on-going, adaptive communication among biological systems in nature, particularly among species in an ecological community. In an ecological community, the member species continually adapt and modify behaviors over time in response to changes in the environment so as to maintain an overall balance of inputs and outputs within the community. In the biorefinery system, the photobioreactor, anaerobic bioreactor, pyrolysis device, and greenhouse space comprise components or “species” within the ecological community that is the biorefinery system. The biorefinery system includes an autonomous control system competent to (1) continually sense and communicate the current behavior of each component in the system and of the system in general, and (2) continually modify and adapt both component behavior and system behavior as needed for evolving changes in inputs and outputs of the system. The control system is competent to discover new methods and combinations for balancing inputs and outputs, learning from the behavior of system components, just as an ecological community does to evolve over time. The biorefinery structure described in detail below brings the members of a particular ecological community into close proximity, and the control system described in detail below accelerates the communication that naturally occurs within an ecological community. In addition to providing a system that generates product without unwanted waste, the system also accelerates the generation of natural products. In nature, it takes about 400 years for a tree to decompose and recarbonize soil, and about 1,000 years for natural processes to make one inch of soil. As described in detail below, the biorefinery system can produce natural, organic carbonized soil and soil products in 30-50 days.

### Definitions

Before describing the biorefinery system 100 of FIG. 1 in greater detail, definitions are provided directed to various components, processes, inputs, and outputs of the biorefinery system 100.

As used herein, the term “biorefinery” or “bioprocessor” describes a facility that integrates one or more biomass conversion processes and equipment to produce fuels, power, heat, and other value-added chemicals or by-products from biomass.

As used herein, the term “biomass” describes biological material from living or recently living organisms and includes, without limitation, all matter produced by plants or other photosynthetic organisms, including plant matter; wood; wood waste; forest residues, including dead trees, branches and tree stumps; yard clippings; wood chips; food waste; algae or algae digestate; photosynthetic micro-organisms and their digestates. Biomass may also include lignocellulosic biomass.

As used herein, the term “lignocellulosic biomass” includes any plant biomass comprising cellulose, hemicellulose, and lignin including, without limitation, agricultural residues such as corn stover or other plant material residue left in a field after harvest; dedicated biomass energy crops; wood residues such as sawmill and paper mill discards, and forest detritus; and paper waste.

As used herein, the term “photosynthetic bioreactor” or “photobioreactor” or PBR” describes a system for cultivating algae, including microalgae, and/or other photoautotrophs or photosynthesizing microorganisms for the purpose of fixing carbon dioxide, and/or producing a carbon-rich biomass. Useful organisms include, without limitation, diatoms and cyanobacteria (also known as blue-green algae), Chlorella, Spirulina, Botryococcus braunii, Dunaliella tertiolecta, Graciaria, Pleurochrysis carterae, and Sargassum, to name a few of the tens of thousands of species currently known to be in existence. In a preferable embodiment, the algae or other photosynthesizing microorganisms may be nitrogen fixing species.

It will be understood by those skilled in the art that useful photosynthesizing microorganisms, including microalgae, can include combinations of named or unnamed species growing in and collected from, local natural or man-made ponds. In one embodiment, useful photosynthetic microorganisms are cultured in the PBR in the presence of biomass, such as lignocellulosic biomass. In another embodiment, the microorganisms are cultured in the presence of spent brewing mash or hops solids, or similar germinated grain compositions. In another embodiment, the microorganisms are cultured in the presence of biochar or organic carbon. In another embodiment, the microorganisms are cultured in the presence of rocks or crystals (whether whole or pulverized as rock powder or rock salt) to provide micro-nutrients, such as minerals and trace elements.

As used herein, the term “anaerobic bioreactor” or “ABR” describes a biomass digestate process or system. Exemplary ABR biomass feedstock may include one or more of the following: the output of a PBR; food waste; and water treatment plant sludge and/or slurry. ABRs designed in accordance with embodiments of the present disclosure may include one or more stages for anaerobic digestion of biomass feedstock to produce both liquid and solid bioenergy products of value.

In one embodiment, the ABR biomass feedstock is algal feedstock, and the ABR output may include one or more of the following products: methane, hydrogen, carbon dioxide, a nitrogen-rich liquid digestate, referred to herein as a digestate liquor, comprising a high-grade organic nitrogenous soil regenerating product suitable for use an agricultural soil amendment or fertilizer; and nutrient-rich algal digestate solids. If the feedstock includes material that is not suitable for agriculture, for example, the sludge or slurry from a treatment plant, the digestate liquor and digestate solid can be used as non-agricultural soil amendments, such as to rebuild forest soils or for use in municipal plantings or other horticultural applications. The ABR methane and hydrogen outputs may be used as feedstock for an energy conversion system, which can be used to convert the methane and/or hydrogen into energy in the form of electricity. The carbon dioxide can be used as a nutrient feedstock for the photosynthetic bioreactor system 104.

As used herein, the term “greenhouse” describes an environment or system that contains at least portions of the PBR and the ABR systems. The conditions in the greenhouse may be optimized so as to be used to grow discrete plant life, separate from the functions of both the PBR and ABR systems.

As used herein, the term “biomass gasifier” or “biomass pyrolysis system” describes a system for thermochemical decomposition of organic material or biomass at elevated temperatures in the absence of oxygen. The output is a porous, stable, carbon-rich product referred to herein as “biochar”, “organic carbon” (because it has been broken down to be substantially elemental carbon), “charcoal” and “active charcoal”. Biochar or organic carbon is a stable, porous solid rich in carbon content and useful for sequestering and locking carbon into the soil, also referred to in the art as atmospheric carbon capture and storage.

As used herein, the term “organic carbon pyrolysis system” describes one embodiment of a biomass pyrolysis device or biomass gasifier of the present disclosure. The temperature of the pyrolysis in the organic carbon pyrolysis device may vary. For example, in one embodiment, biochar or organic carbon compositions are produced by pyrolysis at temperatures of at least 800° F. In another embodiment, organic carbon compositions are generated by pyrolysis at temperatures of less than 1,000° F. In another embodiment, organic carbon compositions useful in this disclosure are produced by pyrolysis at temperature ranges between 800-900° F.

As can be seen in FIG. 1, the outputs from the organic carbon pyrolysis system 102 outputs in accordance with embodiments of the present disclosure are collected and utilized in a closed loop process. In particular embodiments, syngas and bio-oil outputs, including hydrogen and methane, are utilized to (1) power the gasification process itself, and/or (2) comprise feedstock for the energy conversion system; and CO₂ and NO outputs are provided to a PBR as nutrient sources for algal colony growth. In another embodiment, some of the heat generated by the organic carbon pyrolysis device is provided as a heat source to a PBR by means of a closed loop process. In still another embodiment, water vapor output is condensed and utilized as a reclaimed water source for at least one of the following: (1) a PBR system 106; (2) a hydronic heating/cooling system for the PBR system 106 and/or for the greenhouse system 110, and (3) an irrigation source for plant cultivations. Useful feedstock for the organic carbon pyrolysis device includes, without limitation, any woody biomass, including wood waste and hog fuel, macadamia nut shells, weeds, stover, and the like.

Provided below is a description of individual devices, the biorefinery system, and high value bioenergy outputs produced, as well as exemplary, non-limiting examples, which (1) demonstrate the suitability of the components and systems described herein in the methods of the disclosure, and (2) provide descriptions for how to make and use the same.

### Biorefinery System Overview

Referring to FIG. 1, a member device interrelationship in an exemplary carbon-sequestering biorefinery system 100 is shown. Key to the function of the biorefinery system is the ability to utilize its various component outputs efficiently through closed loop processes so that the system is substantially carbon-negative.

The biorefinery system 100 described in FIG. 1 consumes waste heat and carbon dioxide, for example, generated by the pyrolysis of biomass in the biomass pyrolysis system 102. The waste heat and carbon dioxide support the cultivation of energy-rich biomass, such as algae, and its conversion into useful forms. Such systems are ideally suited for the production of methane and hydrogen that can be used as fuel for transportation, farm equipment or converted to electrical power. The system 100 shown in FIG. 1 is designed to produce no waste; rather, its byproducts are valuable high-grade, nutrient dense, organic soil regenerating products, such as fertilizers, soil amendments, and soil regenerating products.

The individual components of the biorefinery system shown in FIG. 1 will now be separately described. After the components have been described, the interrelationships between the individual components in the exemplary biorefinery system will be described in greater detail.

### Photobioreactor

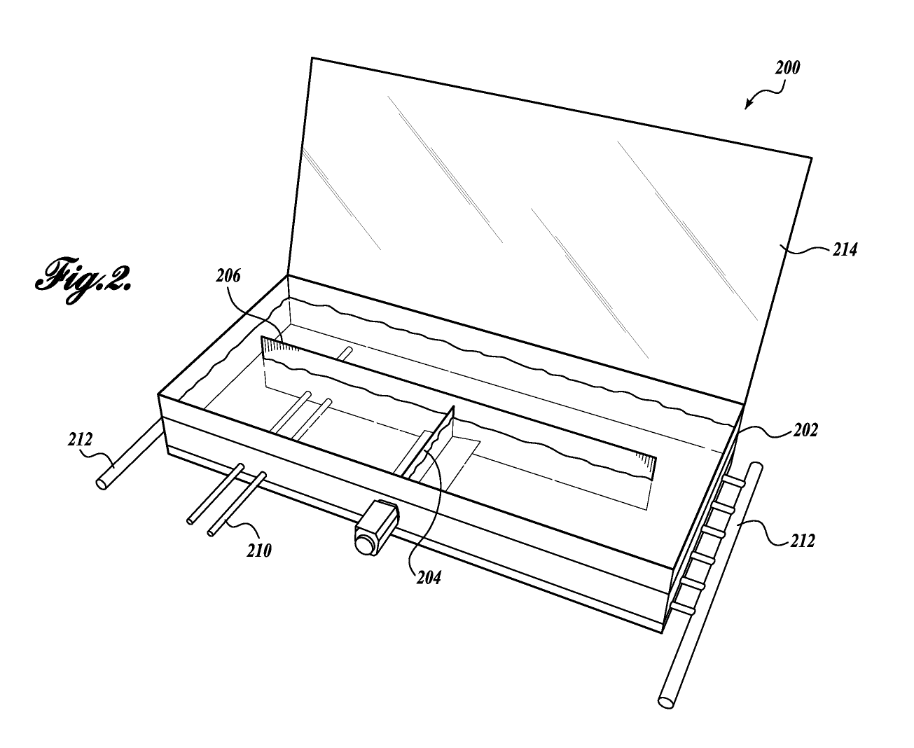

Referring to FIG. 2, an illustrated embodiment of a photobioreactor system 200 is shown. Photobioreactors are essentially growing devices for photosynthetic microorganisms. The photobioreactor 200 in the illustrated embodiment of FIG. 2 includes a raceway 202, and a mixing system, which includes a mixing device 204 and a divider 206. The raceway 202 holds water and therefore provides an aqueous environment in which the photosynthetic microorganisms can be cultivated and harvested. The mixing device 204 is configured to circulate the microorganisms to enhance environment mixing and microorganism growth.

Photosynthetic microorganisms convert sunlight and carbon dioxide into carbon-rich polymers, such as sugars, starches and oils, making them an ideal, natural carbon-sequestering agent. After a growth period, the carbon-rich polymers can subsequently be digested and modified to produce numerous high-value biofuels, including biodiesel and other useful fuels. As a non-limiting example, the microorganisms are one or more species of algae or microalgae. As another non-limiting example, the microorganisms may include other non-algal photosynthetic microorganisms, such as photosynthetic bacteria, for example, cyanobacteria (also known as blue-green algae). In one embodiment, the microorganisms used with process described herein may include nitrogen-fixing species.

For simplification in the disclosure, photosynthetic microorganisms will be generally referred to herein as “algae”, even though suitable photosynthetic microorganisms may include bacteria that behave like algae. The utility of algae, as well as general descriptions for how to grow the algae and convert the product into biofuels, is well documented in the art. As mentioned above, the inventors have found that suitable photosynthetic microorganism species for an exemplary working system include diatoms and cyanobacteria, Chlorella, Spirulina, Botryococcus braunii, Dunaliella tertiolecta, Graciaria, Pleurochrysis carterae, and Sargassum, etc.

Different algal species have different growth requirements, and a given species may have different growth requirements depending on the time of day (or night) and/or the time of year; the quantity and quality of nutrients, minerals, and other components present in the growing environment, the water temperature, sunlight levels, and/or the density of the algal population. PBRs in accordance with embodiments of the present disclosure may provide means to manage and modulate growth conditions, provide continual or periodic feedstock inputs of algae, sun, carbon dioxide and/or other desired growth enhancing agents.

A PBR typically has means for modulating the water supply temperature because most algae have preferred growing temperatures. If the PBR gets too cold, the growth of the algae slows; if it gets too hot, the algae die. PBRs, and particularly the raceways in which the algae grow, can be heated by any means including using waste heat provided from one or more member devices in a biorefinery system (see, e.g., FIG. 1), as will be described in greater detail below. A suitable temperature range for an exemplary photosynthetic microorganism, such as cyanobacteria, is in the range of about 50 F to about 120 F, alternatively in the range of about 50 F to about 85 F, and alternatively in the range of about 65 F to about 80 F.

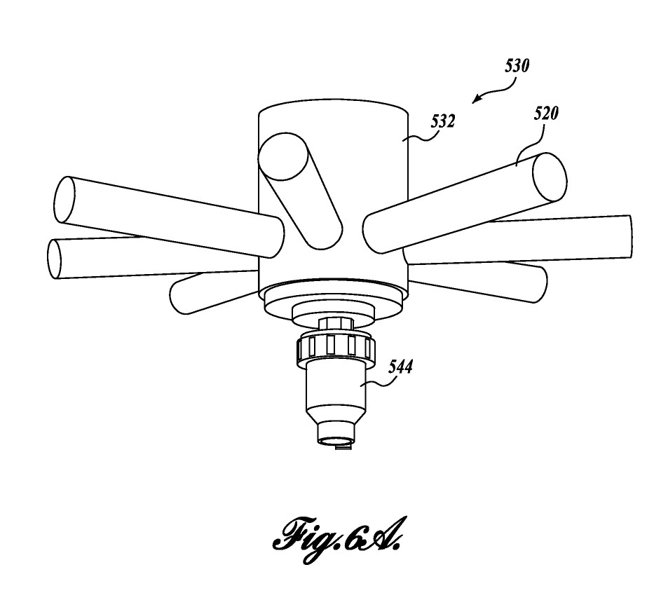

Alternatively, temperature modulation can be provided by thermally heated or cooled air or water. In a non-limiting example, well water or ground water can be collected and heated by the biorefinery system, for example, by utilizing the thermal output of the biomass pyrolysis element, for example, provided to the PBR by means of a hydronic radiant floor system. In another embodiment, the water utilized in the hydronic system includes condensed water vapor collected from the biomass pyrolysis system 102. In another non-limiting example, geothermally heated or cooled air is provided by means of earth tubes that utilize the earth's own geothermal energy to raise or lower the ambient temperature as desired. Exemplary earth tubes 550, as described in greater detail below, are shown in the illustrated embodiment of FIG. 5.

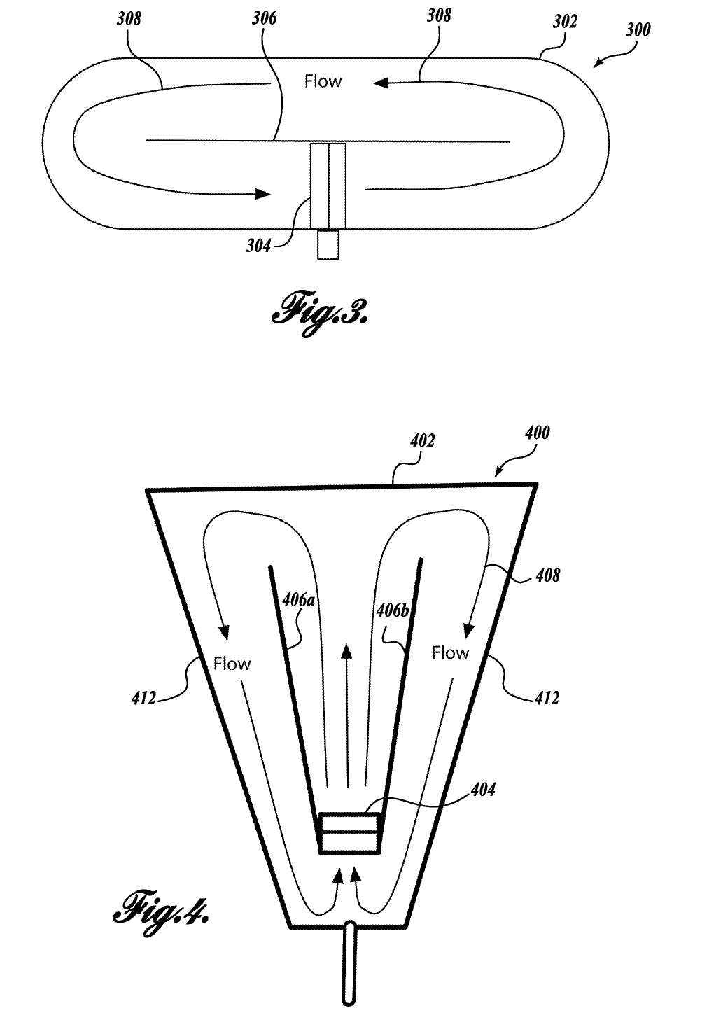

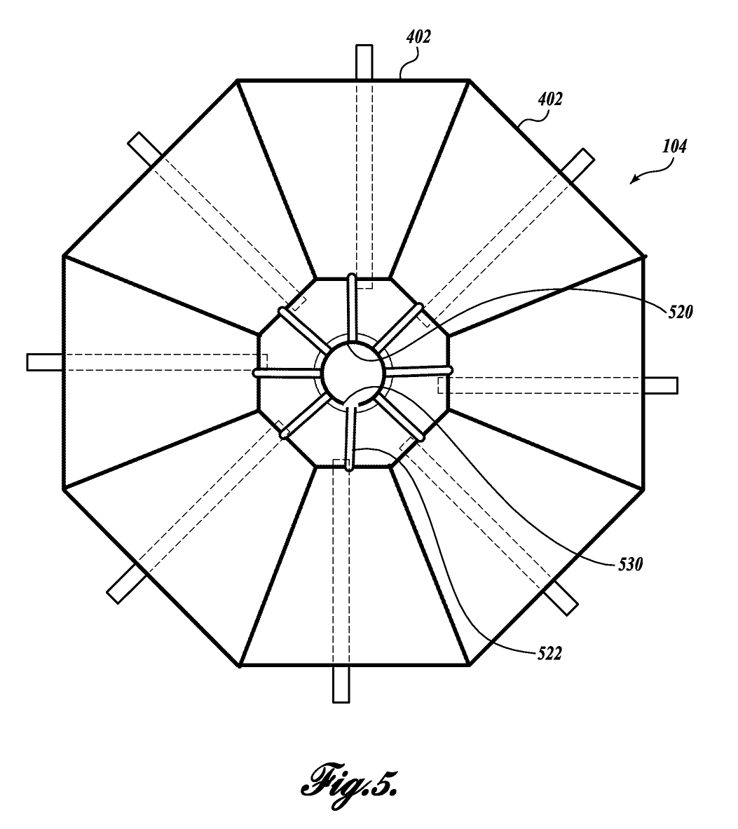

Returning to FIG. 2, the raceway 202 in the illustrated embodiment is a substantially rectangular, horizontal container for growing algae, however, it should be appreciated that the raceway may be designed to be vertical, horizontal, tubular, or in any other suitable configuration. As non-limiting examples, FIGS. 3 and 4 illustrate alternative raceway designs, for example, a rectangular raceway 302 with rounded ends and a trapezoidal raceway 402, respectively. It should be appreciated that the raceways 302 and 402 shown in FIGS. 3 and 4 are substantially similar to the raceway 202 of FIG. 2, except for differences regarding their shape and fluid flow dynamics. Like part numerals are used in FIGS. 3 and 4 as used in FIG. 2, except in the 300 and 400 series.

In the illustrated embodiment of FIG. 2, the raceway 202 has a center divider 206, with the mixing device 204 (shown as a motorized paddle wheel) positioned on one side of the divider 206. This configuration allows for a fluid path in the raceway 202 around the divider 206 (whether clockwise or counterclockwise, depending on the turning direction of the mixing device 204). (See, for example, the fluid flow path shown in the illustrated embodiment of FIGS. 3 and 4, depicted by respective sets of arrows 308 and 408).

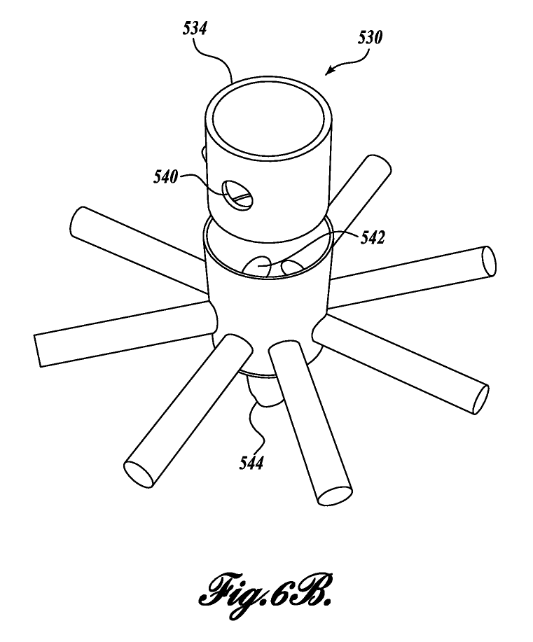

The raceway 202 may be sloped toward one end to facilitate drainage of the raceway 202 to a drain hole (not shown) during algal harvest. As described in greater detail below, the algal harvest may be drained into a concentrator tank 510 (see FIGS. 5 and 6). As seen in FIG. 2, the raceway 202 may include a lid 214, such as a transparent polycarbonate lid; however, such a lid is not necessary, and an open or partially open raceway 202 is also within the scope of the present disclosure.

Constant fluid flow in the raceway with minimized dead spots is desired to create a healthy algal growth environment. Referring to FIG. 3, the raceway 302 has been optimized for fluid flow 308 with rounded ends that discourage dead spots. Referring to FIG. 4, in a substantially trapezoidal shaped raceway 402, the inventors found that a configuration with a single divider created fluid flow dead spots in the raceway 402. Therefore, the fluid dynamics of the trapezoidal shaped raceway 402 were improved by including two dividers 406a and 406b, with the mixing device 404 (shown as a motorized paddle wheel) positioned between the two dividers 406a and 406b. In the illustrated embodiment of FIG. 4, the dividers 406a and 406b are oriented to be substantially parallel with the sidewalls 410 of the raceway 402. The result is a mixing pattern that flows in two fluid paths that start inside the dividers 406a and 406b and flow outwardly toward the sidewalls of the raceway 402, as indicated by arrows 408.

Mixing in the PBR promotes a healthy algal growth environment, and can also be used to harvest the algae in the PBR. In the illustrated embodiments, mixing is achieved by the mixing devices, which may be paddle wheels or other suitable mixing devices. It should be appreciated that the mixing device may be configured and controlled to operate at different speeds, for example, steady state and harvest conditions. Moreover, if the control system senses frictional force on the mixing device, the control system may control the mixing device to speed up and/or reverse direction for a period to break up any material in the PBR that may be clogging the mixing device. In one embodiment of the present disclosure, mixing is at a steady state during the algal growth state; but during harvest, the mixing is increased to lift the algal sediments from the bottom of the raceway.

Referring to FIG. 2, the raceway 202 further includes a gas bubbler 210 for bubbling carbon dioxide, air, nitrogen, and/or other gases into the water in the raceway 202. Carbon dioxide, normally considered a pollutant, is used as a nutrient for the algae. In addition to carbon dioxide, nitrogen and other gases may also be bubbled into water in the raceway 202 as nutrients for the algae. Carbon dioxide may be received from one or more other systems, for example, a biomass pyrolysis system, an energy conversion, system, an anaerobic bioreactor system, or a flue gas, for example, from an industrial furnace, such as a wood mill or coal furnace. As one non-limiting example, one source of nutrient gas may be to combust a syngas output from the biomass pyrolysis system 102 (see FIG. 1) to harness the energy from such combustion, and then to bubble the combusted gas into the water in the raceway 202. In addition to gases, a portion of the nitrogenous fertilizer output the anaerobic bioreactor may also be used as a nutrient for the algae.

The feedback for rate of flow of gases (such as carbon dioxide) and other nutrients to the raceway 202 via a gas bubbler 210 may be, for example, the pH of the water in the raceway 202 and, if the PBR is contained in the greenhouse 110 (see FIG. 1), the carbon dioxide level. Either one or both of these parameters may be indicative of excess or inadequate carbon dioxide (and other nutrients) being bubbled into the PBR 200.

Horizontal raceway PBRs designed in accordance with embodiments of the present disclosure may be large ponds that rely on solar energy and the ambient temperature of the environment to sustain the algal growth. In accordance with embodiments of the present disclosure, heat exchangers 212 can be used to regulate the temperature of the raceway 202 to enhance algal cultivation. As described in greater detail below, the heat exchangers 212 may be configured to harness unwanted heat outputs from other components and processes (for example, the biomass pyrolysis system 102) in the biorefinery system 100. In one embodiment, the heat exchangers are part of a hydronic radiant heating/cooling system.

A control system may be used to continuously monitor and adjust multiple environmental parameters to maximize the algal rate of growth. For example, the heat exchangers 212 may be controlled to mimic the natural diurnal rhythms of the algae. Typically, growth rates increase when the temperature varies between 80° F. during the day and 65° F. at night. Because higher temperature reduces the solubility of gases in water, the growth cycle may be related to a natural breathing cycle of the algae.

Referring now to FIG. 5, a multiple PBR system 104 is shown including multiple trapezoidal raceways 402, as can be seen in FIG. 4. In the multiple PBR system 104 shown, the trapezoidal raceway design is selected to optimize the surface area, and therefore, the volume of the PBR system, when multiple PBRs are joined in a parallel system having a center algal collection and concentration tank 520. However, it should be appreciated that rectangular raceways 202 and 302, such as those shown in FIGS. 2 and 3 may also be used in a multiple PBR system. In the illustrated embodiment, the system 500 includes eight raceways 402; however, it should be appreciated that a suitable system may be designed with any number of raceways.

In the illustrated embodiment, the raceways 402 are configured in a polygonal configuration, each having a side adjacent the valve system 530, as described in greater detail below.

One advantage of a multiple PBR system is that a fraction of the algae in the total system can be collected and concentrated over a period of time during the growing cycle. For example, if the growing cycle is about 8 days, the system can be designed such that one PBR may be drained each day to a collector tank to provide a batch-continuous system. Moreover, a multiple PBR system also allows for experimentation in the system because different algae can be grown in individual PBRs, and/or different operations conditions can be set in individual PBRs to experiment with and optimize the different growing conditions for the algae. It should be appreciated that the configuration of the raceways 402 in FIG. 5 may provide the base for a greenhouse 110, as described in greater detail below.

The raceways 402 in the illustrated embodiment of FIG. 5 are preferably oriented to be sloped toward the center of the polygon to facilitate drainage of the raceway 202 during algal harvest. In the illustrated embodiment, the raceway 402 may be drained into an algal concentrator tank 520 positioned in the center of the plurality of raceways 402. In that regard, each raceway 402 has a raceway drain 522 that leads from the raceway to the concentrator tank 520.

A selector valve system 530 is configured to select one of the raceway drains at any given time. Referring to FIGS. 6A and 6B, the valve system 530 generally includes an outer shaft 532 and an interior shaft 534 that rotates relative to the outer shaft 532. The interior shaft 534 has a hole 540 that aligns with holes 542 in the outer shaft 532 positioned at the respective raceway drains 522. Therefore, the interior shaft 534 rotates to align its hole 540 with a raceway drain 522 to select the raceway 402 that will be harvested. When aligned, a harvest valve 544 may be activated to allow the raceway 402 colony to flow into the concentrator tank 520.

In the illustrated embodiment of FIG. 5, the raceway 402 at six o'clock is selected and is draining through raceway drain 522 and valve 530 into the concentrator tank 520. If each raceway is configured for harvest after about 24 hours, then the system can be configured to cycle every 8 days.

It should be appreciated that the valve system 530 may include a motor (not shown) to rotate the interior shaft 534 relative to the outer shaft 532. In one embodiment of the present disclosure, the individual raceway drains 522 are indexed using a Hall Effect device that senses when the hole 542 in the interior shaft 534 is aligned with the hole 540 in the raceway drain 522. Alternatively, the motor (not shown) may be a stepper motor that is programmed to travel a precise number of steps to index the hole 542 in the interior shaft 534 with the hole 540 in a subsequent raceway drain 522.

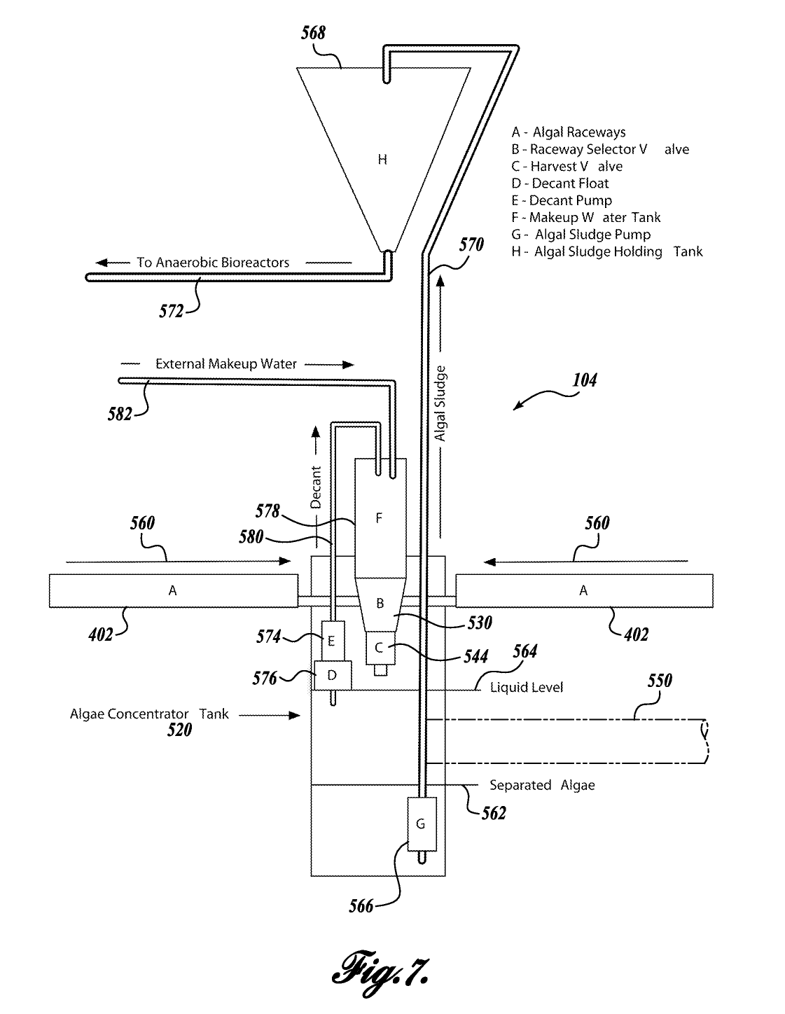

Referring to FIG. 7, a cross-sectional view of the PBR system 104 is shown. The system 104 includes an algal concentrator tank 520 that receives algal discharge from each of the raceways 402, as can be seen in FIG. 5. Arrows 560 indicate the flow of the discharge from the individual raceways 402. As discussed above, the illustrated PBR system 104 is designed to process the discharge of one raceway 402 at a time. In other embodiments, however, the PBR system 104 may be configured to process the discharge of more than one raceway 402 at a time. When the raceway selector valve 530 (see FIGS. 5, 6A, and 6B) is positioned to select a specific raceway 402, the harvest valve 544 is opened, and the raceway 402 contents are discharged into the concentrator tank 520.

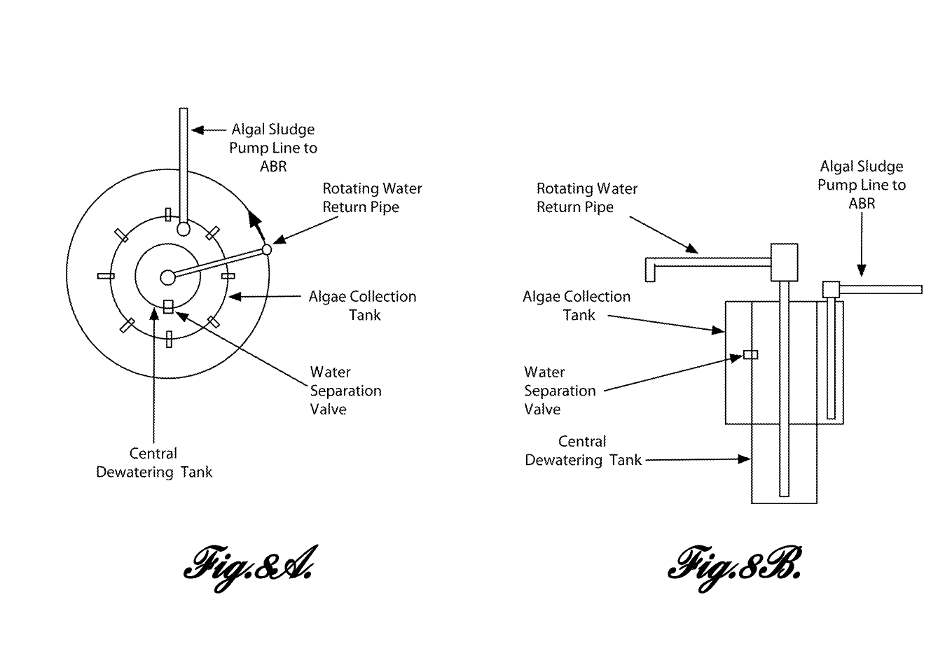

When the algal discharge is received in the concentrator tank 520, there is no mixing and the harvest is left to decant. In that regard, the algal sludge separates and sinks to the bottom of the tank, while the water rises to the top of the tank, as indicated by respective lines 562 and 564 in the concentrator tank 520. In the illustrated embodiment, a pump 566 pumps the algal sludge to a holding tank 568 by line 570, and then to the anaerobic bioreactor system 106 (see FIG. 8) by line 572 for further processing, as will be described in greater detail below. In accordance with one embodiment of the present disclosure, the collected algal harvest is decanted for a period of about 24 hours.

In the system configuration shown in FIG. 7, the holding tank 568 is vertically offset from the PBR, thereby requiring a pump to move the algal sludge upward to the holding tank 568. However, it should be appreciated that in other systems, the anaerobic bioreactor is positioned below the raceways so that a pump is not required and gravity assists the travel of the algal sludge to the ABR holding tank.

After decantation, the decanted water may be recycled and reused in the emptied raceway 402. In that regard, a decant pump 574 is positioned on a float 576 to float on the top of the decant water level. There, the pump 574 pumps water to a makeup water tank 578 through line 580, which refills at least one of the raceways 402 via the raceway selector valve 530. In addition to decanted water, an external water source may also add water to the makeup water tank 578 via line 580.

In the illustrated embodiment, the makeup water tank 578 is positioned about the raceway selector valve 530. Therefore, the force of gravity will deliver water from the tank 578 to the selected raceway 402 when the valve is open. In another embodiment of the present disclosure, the makeup water tank 578 may refill the raceways 402 with water via another line besides the raceway selector valve 530, for example, using a pump and a rotating water return pipe, as shown in the alternate embodiment in FIG. 8.

In accordance with embodiments of the present disclosure, a control system can be used to control the functions of the PBR. For example, the control system may be used to:

1. Regulate the speed and direction of a mixing device (or paddle wheel) that circulates the algae in the raceway and mixes gases and nutrients into the raceway water. Prior to harvesting, the paddle wheel speed is increased to bring algae that have settled to the bottom of the raceway into suspension prior to opening the drain;

2. Regulate the flow and the mixture of carbon dioxide and nitrogen (air) through the bubblers;

3. Open and close the drain that carries the algae to the concentrator tank, and subsequently to the ABR for digestion; and/or

4. Regulate the flow of hot water through the heat exchangers to control the raceway temperature.

The approach of the multi-raceway PBR system 102 shown in FIG. 5 is to use multiple small PBRs and harvest a small amount (e.g., one-eighth) of the total algal population frequently. However, it should be appreciated that larger, unmodulated PBRs may also be within the scope of the present disclosure. The advantage of multiple smaller PBRs is greater control over the growth rate within an array of PBRs rather than the total amount of algae accumulated in a single raceway, providing greater sensitivity for the needs of the system, greater control of energy expenditure within the system, and a wider range of options for choosing solutions that support optimal output for an integrated biorefinery system.

Returning to FIG. 5, in addition to hydronic system heat exchangers, earth tubes 550 may be positioned under the raceways to also act as heat exchangers for the PBRs. The earth tubes are buried under the front line, with one terminus external to the biorefinery enclosure (or greenhouse 110) and the other one internal. In FIG. 5, the earth tubes terminate in an air exchange zone (not shown) in the center of the raceway array. In colder weather, cold air is pulled into the earth tubes 550 from outside by passive convection, and the cold air is warmed as it traverses the earth tubes 550, also warming the PBR raceway above it. As the warm air enters the exchange zone at the center of the array, it rises, warming the ambient air in the greenhouse 110 (see FIGS. 1 and 2), which in turn supports maintaining optimal PBR growth temperatures. The greenhouse 110 also may have a ceiling screen that can be activated to effectively lower the greenhouse ceiling, thereby supporting a faster recirculation of ambient air in the greenhouse 110. The greenhouse 110 may also have a fan or vent system to also support faster recirculation of ambient air in the greenhouse 110.

In warmer weather, the air in the earth tubes 550 is cooled geothermally, and the process is reversed. Cooled air terminates at the exchange zone, pushing warmer air up and increasing circulation and ambient air cooling. It will be appreciated by those skilled in the art that the interior earth tube termini may be at ground level, or may extend vertically some distance.

Thus, the ground under the greenhouse 110 acts as a thermal battery or thermal storage unit. In the case of the hydronic heat exchange system, the ground is a thermal battery for heat output generated by member devices in the systems described herein. This heat may be available to the PBRs and greenhouse itself, as desired.

As described above, additional agents can be added to the raceway colony to enhance algal growth. As non-limiting examples, suitable agents include is lignocellulosic biomass, pyrolized carbon (as described in greater detail below), waste mash from brewery production, germinated rice, other grain mash, etc. Placing the agent in a perforated container in a corner of the raceway, for example, is sufficient as the paddle activity will introduce the materials into the raceway over time. Preferred quantities of agent will vary depending on the algae species, raceway volume, and agent composition. As a non-limiting example, for a 70 sq. ft. raceway with water at a depth of 4 inches, the inventors found that the addition of 2-4 cups of agent has a positive impact on microalgal growth, particularly when the algae colony includes Chlorella and/or Spirulina species.

When lignocellulosic biomass, such as wood chips, or organic carbon is used as an agent in the PBR, the material is preferably sized so that it becomes part of the dewatering system later on. In that regard, algae have a tendency to attach themselves to the cellulosic or carbon material. The advantage of such attachment is that the algae stays suspended in the raceway 402 and has less of a tendency to mat. Continued suspension helps the algae receive light, thereby improving its growth rate. As an alternative to the concentration tank 520, shown in FIG. 5 for separation of the algae and water, the algal discharge from the raceway 402 may instead pass into a large strainer with holes after the control system opens the drain 522. In that regard, the holes in the strainer may be sized such that most of the algae and agent are held back as the water (and some algal population) is pumped back into the PBR to begin a new batch. Circulating the water immediately back into the PBR conserves heat and flushes more of the algae and cellulose from the tank because the drain remains open for a longer period of time.

Lignin and hemicellulose in wood take a long time digest anaerobically, but the high nitrogen content of algae can be used to break down the lignin and hemicellulose prior to digestion. Mixing cellulosic materials with algae increases the methane yield from the ABR, as discussed in greater detail below. The inventors found algae also attach well to pyrolized carbon, as compared to unpyrolized cellulosic materials. In addition, mixing pyrolized carbon as an additive in the PBR plays a role in aiding in digestion in the ABR. In that regard, cellulosic materials tend to slow down the digestion process of the algae because the cellulosic materials also need to be digested; however, pyrolized carbon generally does not require digestion because of its elemental form.

The operation of the PBR system 104, as seen in FIGS. 5 and 7 will now be described in greater detail. To start the system, raceways 402 may be filled with water and algae, and other optional agents may be added. Water can be recycled through the system, for example, from the algae concentrator, or added to the system by another water source. In addition cellulosic biomass or lignocellulosic biomass may be added to the system as a nutrient for the algae. Other nutrients may also be added. Carbon dioxide is bubbled through the raceway bubbler (not shown in FIG. 5, but see bubbler in illustrated embodiment of FIG. 2). In addition, other gases may also be bubbled, such as syngas, nitrogen, or air.

After inoculation, the PBR raceway 402 is allowed to cultivate for a specified period of time. During this time, the mixing device 404 (see FIG. 4) mixes the raceway 402 slowly and constantly, at a non-limiting example of a rate of about less than 10 rpms. If the mixing device 404 gets clogged, the user or the control system may detect the clog and provide either reverse mixing or speed up the mixing to break up the clog

When a pronounced decrease in the algal growth rate is detected, either by control or after a specific cultivation time period, the harvesting sequence is initiated and the biomass is moved to the next stage of processing. In one embodiment of the present disclosure, the raceways 402 are configured to be ready for harvest after about 24 hours. In another embodiment of the present disclosure, the raceways are configured to be ready for harvest in a range of about 1 to about 8 days, more preferably about 3 to about 8 days, and even more preferably about 5 to about 8 days.

As a non-limiting example, the PBR control system may be configured to sense the density of the algae. When the density reaches a certain point where the light penetration into the raceway is reduced, resulting in a slower rate of growth, the control system may open the drain at the bottom of the PBR and increase the speed of the mixing device to move the algae from the raceway 402 to the concentrator tank 520. As a non-limiting example, when harvesting, the mixing device may move at a rate of up to about 30 rpms.

After dewatering, most of the separated liquid is pumped back into the PBR to retain the heat and residual nutrients to begin the next batch of algae. The algal-cellulosic feedstock is pumped into the a holding tank 568 (see FIG. 7) to initiate the hydrolysis stage of the ABR (the breakdown of organic polymers—proteins, carbohydrates and lipids—into organic monomers—amino acids, sugars and fatty acids) and begin the conversion into methane, hydrogen, and nitrogenous soil regenerating and fertilizing products. Hydrolysis is a preparation stage for anaerobic digestion, which is performed in the anaerobic bioreactor (see FIG. 1).

As another non-limiting example, when a certain algal density is reaches, the PBR control system may stop the mixing device 404, stop the flow of carbon dioxide and nitrogen in the bubblers, and increase the raceway temperature to above 85° F. Deprived of nutrients and exposed to excessive heat, the algae begin producing more lipids and then shortly thereafter they begin to die.

If left in this state for 1 or 2 days, the algal substrate begins to undergo hydrolysis in the raceway 402. In a system such as the one illustrated in FIG. 5, where there are multiple raceways arranged in an octagonal array, different raceways may have algae in different stages of growth. Therefore, temporarily using a PBR as part of the digestion process may increase the rate of digestion without impeding the rate of algal production.

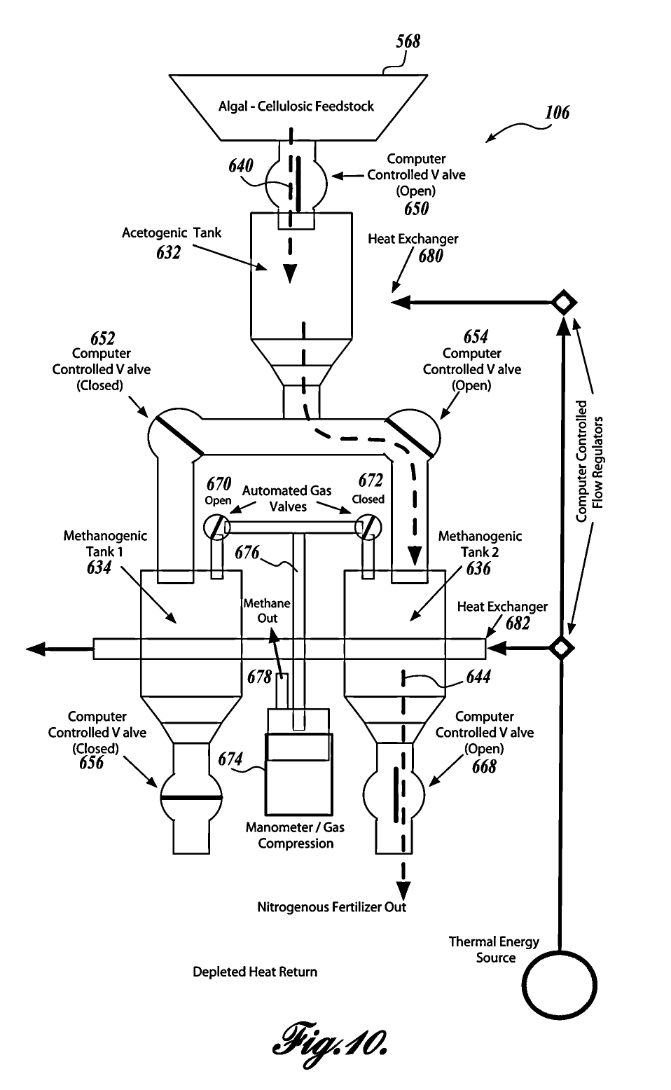

After 1 or 2 days, the control system turns the mixing device 404 back on and runs it at high speed to lift the settled algae and cellulose into suspension. The control system then opens the drain in the bottom of the PBR to move the algae into the collection tank 520 for dewatering. Most of the separated liquid is pumped back into the PBR to retain the heat and residual nutrients to begin the next batch of algae. After dewatering in the concentrator tank 520, the algal-cellulosic feedstock can be pumped directly into the acetogenic stage 632 (see FIG. 10) of the ABR to complete its conversion into energy products and fertilizing and soil regenerating products.

### Anaerobic Bioreactor

Returning to FIG. 1, an anaerobic bioreactor or “ABR” system 106 is shown as a component in the biorefinery system 100. In general, ABR systems are configured to digest organic material in an anaerobic environment, using one or more microbial species. The choice of organic feedstock and bioenergy product outputs desired will inform both the choice of anaerobic microorganisms utilized and the number of stages for the ABR. The number of stages in a given ABR reflects the need for different local environments that support optimal microbial digestion.

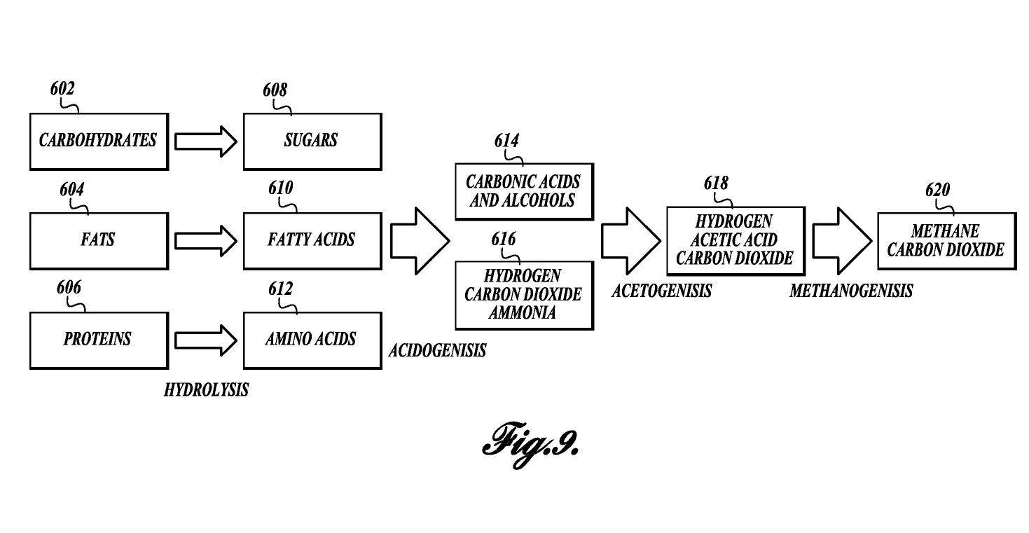

In the illustrated embodiment of FIG. 1, the ABR 106 is configured to primarily digest algal feedstock, which is an output from the PBR 104. Referring to FIG. 9, a flow chart for the digestion of an algal feedstock is provided, where methane and hydrogen are desired bioenergy output products. The digestion process starts with hydrolysis, which is the conversion of carbohydrates, fats, and proteins, indicated by blocks 602, 604, and 606 to sugars, fatty acids, and amino acids, indicated by blocks 608, 610, and 612. The process of hydrolysis may takes place, for example, in the raceways 402 (see FIG. 5) or in a holding tank 568 (see FIG. 7).

After hydrolysis, the material from hydrolysis (i.e., sugars, fatty acids, and amino acids, indicated by blocks 608, 610, and 612) typically is subjected to an acidogenesis process to form carbonic acids and alcohols, hydrogen, carbon dioxide, and ammonia, indicated by blocks 614 and 616. Alternatively, hydrolysis and acidogenesis may occur concurrently, for example, in a single tank.

After acidogenesis, the material from acidogenesis (carbonic acids and alcohols, hydrogen, carbon dioxide, and ammonia, indicated by blocks 614 and 616) is subjected to acetogenesis to form hydrogen, acetic acid, and carbon dioxide, indicated by block 618. The hydrogen gas may be collected as an energy product for the energy conversion system. The carbon dioxide may be collected as feedstock for the PBR system.

After acetogenesis, the material from acetogenesis (algae digestate and acetic acid, indicated by blocks 618) is subjected to methanogenesis to form methane and carbon dioxide, indicated by block 620. Methane gas may be collected as an energy product for the energy conversion system. The carbon dioxide may be collected as feedstock for the PBR system.

Useful, benign, and environmentally safe microbial species for digestion are readily available. Specific microbial products may include a number of bacterial species that perform different steps in the digestion of the input feedstock.

Acetogenesis typically occurs through three groups of bacteria: homoacetogens; syntrophes; and sulphoreductors. Exemplary species include Clostridium aceticum; Acetobacter woodii; and Clostridium termoautotrophicum.

Exemplary methanogenic bacteria include Methanobacterium bryantii, Methanobacterium formicum, Methanobrevibacter arboriphilicus, Methanobrevibacter gottschalkii, Methanobrevibacter ruminantium Methanobrevibacter smithii, Methanocalculus chunghsingensis, Methanococcoides burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi & Methanogenium bourgense); Methanoculleus marisnigri, Methanofollis liminatans; Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophila, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus, (Methanobacterium thermoflexum), Methanothermobacter wolfei (Methanobacterium wolfei), Methanothrix sochngenii.

ABRs described herein may be used in a biorefinery system, for example, the biorefinery system 100 shown in FIG. 1, or may be used as stand-alone devices or in other systems to digest other feedstock. Other exemplary feedstock that could be used include the sludge or slurry form water treatment plants and/or waste management plants. Alternatively, the feedstock could come from any plant, mill, or industry comprising organic waste material competent to be anaerobically digested. The exemplary algal feedstock described herein may produce products that are suitable for agricultural applications. However, when the source of the feedstock is an industrial or municipal waste source, the products from these feedstocks would be generally used for non-agricultural applications, such as forest remediation or non-food horticultural applications.

In some applications, ABRs use smaller tanks with distributed processing and load balancing to reduce retention time and increase throughput. In that regard, the ABR system is scalable so more reactor stages can be easily added as energy and soil production demands grow or as the volume of the organic feedstock stream increases.

Referring to FIG. 10 an exemplary anaerobic bioreactor system 106 is shown. The reactor employs a two-stage digestion, the acetogenic stage (indicated by tank 632) and the methanogenic stage (indicated by parallel tanks 634 and 636). Bacteria in the acetogenic stage break down the algal feedstock into the precursors (shown in FIG. 10) that are used by the methanogenic stage bacteria to produce methane. It should be appreciated that the feedstock to the anaerobic bioreactor system 106 may be algal feedstock, or may be mixed with additives that have been added to the algae in the PBR, for example, cellulosic materials, pyrolized carbon, or mash, as discussed above.

Returning to FIG. 7, algal sludge is pumped from the algae concentrator tank 520 to the algal sludge holding tank 568, which may also serve as a hydrolysis tank to complete the first stage of digestion, the conversion of carbohydrates, fats, and proteins, indicated by blocks 602, 604, and 606 to sugars, fatty acids, and amino acids, indicated by blocks 608, 610, and 612, as shown in FIG. 9. It should be appreciated, however, that separate holding and hydrolysis tanks are also within the scope of the present disclosure.

In the illustrated embodiment, ample water remains in the concentrated feedstock that exits the concentrator tank 520, so that it can be pumped from the concentrator tank 520 to the holding tank 568.

After the feedstock has been pumped to the collection tank 568, the flow of biomass through the ABR system 104 is primarily driven by gravity. Because the methanogenic stage takes about twice as long as the acetogenic stage, two methanogenic tanks 634 and 636 are used in parallel (per one acetogenic tank 632) to keep the process running continuously. Sensors for pH in the acetogenic tank 632 indicate the timing for moving the contents from the acetogenic tank to one of the lower methanogenic tanks 634 or 636. The methanogenic tank 634 or 636 that is being loaded from above also releases its contents (containing the liquid and solid fertilizers) via line 644 into a collection area below the ABR (not shown).

Temperature control is important in an ABR system 106 for the rapid digestion of the algal or another microorganism mixed with cellulose in a feedstock blend that comes from the PBRs. The feedstock is at least at ambient temperature, and preferably, warm as it moves from the PBR to the ABR. Ambient to warm temperature is preferred because the acetogenic bacteria tend to work best at about 70° F. There is some heat loss in the dewatering process but the feedstock arrives in the collection tank warm enough to be brought quickly up to temperature. Heat rising from the first stage tank brings the feedstock to the optimal temperature. Each tank uses a separate computer controlled heat exchanger to maintain and vary the temperatures as needed.

Referring to FIG. 10, the path of the feedstock is indicated by arrows 640, 642, and 644. Arrow 640 shows feedstock moving from the collection tank 568 (which may also be a hydrolysis tank) into the acetogenic stage tank 632. Arrow 642 shows the contents of the acetogenic stage tank 632 moving into the right hand methanogenic stage tank 636. Arrow 644 shows the contents of the right hand methanogenic stage tank 636 moving out into the fertilizer processing area where the liquids and solids are separated. The next output from the acetogenic stage tank 632 will move into the now empty right hand methanogenic stage tank 636, while the full left hand methanogenic stage tank 634 prepares for unloading its contents.

Multiple valves 560, 562, 564, 566, and 568 are employed to control the path of the liquid feedstock through the ABR system 106. The valves are preferably computer controlled by an intelligent control system. In addition, a methane off-gas can be purged and collected from the methanogenic stage tanks 634 and 636. Valves 570 and 572 control the flow of the off-gas to a manometer or gas compression tank 674 via line 676, which is then configured to supply methane gas via line 678 to other components in the biorefinery system 100. Carbon dioxide may also be an off-gas, which As shown in the illustrated embodiment, heat exchangers 680 and 682 may be employed to control the temperatures of the various tanks 632, 634, and 636.

The preferred retention times for each tank is the ABR is as follows.
Hydrolysis Tank: The feedstock can be held for up to about 5 days at a temperature in a range between about ambient temperature (about 70 F to about 75 F) and about 95 F.
Acetogenic Stage Tank: The feedstock can be held for about 4-14 days, and more preferably about 5-10 days, and even more preferably about 5-8 days, at a temperature in the range of about 70° F. to about 95 F, or in the range of about 75 F to about 90 F; then it is dropped into one of the second stage tanks, depending on which one was loaded last.
Left Hand Methanogenic Stage Tank—The feedstock can be held for about 8-21 days, and more preferably about 9-18 days, and even more preferably about 10-14 days at temperatures between 125° F. and 135° F., or in the range of about 127 F to about 133 F. The temperature is raised slowly over a period of about 2 days from the temperature in the acetogenic stage to the higher range. The higher temperatures kill the acetogenic bacteria while creating an environment ideal for the methanogenic bacteria to proliferate.
Right Hand Methanogenic Stage Tank—The feedstock is also held for the same time period at the same temperatures as the left hand second stage tank.

Therefore, the total retention time in the ABR, from hydrolysis tank through methanogenesis tank, for a single batch is about 18-40 days, and preferably about 20 days. Retention time through the acetogenic and methanogenic stages (without hydrolysis) is about 13-35 days, preferably about 15 days. In accordance with one method, the acetogenic stage tank has a retention time of about 5 days, and the retention times of each of the methanogenesis stage tanks may be staggered by about 5 days, such that as one tank is at peak methane production the other is ramping up production. When the production rate of one of the methanogenic stage tanks begins to fall off the acetogenic stage tank is ready to replenish the methanogenic stage tank.

Although shown as a separate hydrolysis step, it should be appreciated that the hydrolysis step may begin in the PBR before the harvesting and dewatering functions or may take place in a separate hydrolysis tank, as described in greater detail above. Combining and overlapping the PBR and ABR functions provides a unique and useful improvement over known systems, and highlights the value of an integrated, intelligent cooperative biorefinery system.

A control system may be implemented to regulate the function of the ABRs. For example, temperature, pH, input, and output data may be regulated by the digital control system (DCS) to accelerate the digestion of algal-cellulosic feedstock. The control system is configured to open and close appropriate valves to move the digestate through the system at the appropriate times. The control system may also control and monitor the flow of methane gas from the methanogenic stage in the ABR into a manometer or gas compression tank for storage. The methane collected may be held and compressed for delivery, for example, to the fuel cells (or micro turbines) that may convert it into electrical power. The control system may similarly control and monitor the flow of hydrogen from the acetogenic stage.

### Greenhouse System

In one embodiment of the present disclosure the biorefinery system is a greenhouse system. Returning to FIG. 1, the PBR and ABR systems 104 and 106 can be contained in a substantially closed environment to create a green house biorefinery system 110 that can be used to grow plant life. In that regard, waste heat generated by the system “powers” or heats the greenhouse itself, and the windows providing sunlight to the raceway and raceway configuration that supports algal growth in the PBR array can be cooperatively utilized as space for growing plants for agricultural and/or horticultural applications. Heat sources may include an external heat source, a hydronics system, or a geothermal heat source.

In addition, the high-grade nitrogen fertilizer and nutrient-dense soil regenerating materials produced in this biorefinery provide an ideal growing substrate to produce high-quality, healthy plants. Moreover, plant life irrigation water may be received from reclaimed water in the biomass pyrolysis system 102, described in detail below.

As an example, a biorefinery such as is illustrated in FIG. 1, utilizing mill and logging waste at a lumber or wood-processing plant, for example, can be incorporated into a closed loop system that recovers waste heat and carbon dioxide, as well as other outputs in the system, to (1) sequester carbon and waste heat; (2) generate at least about 1200 kW/day or sufficient energy to manage the energy needs of about 50-100, preferably 75, homes; and (3) generate high value byproducts that provide additional revenue streams, including organic nitrogen-rich fertilizer, organic, nutrient-dense topsoil material, organically-grown plants, and food products derived from these plants.

### Example—Greenhouse System

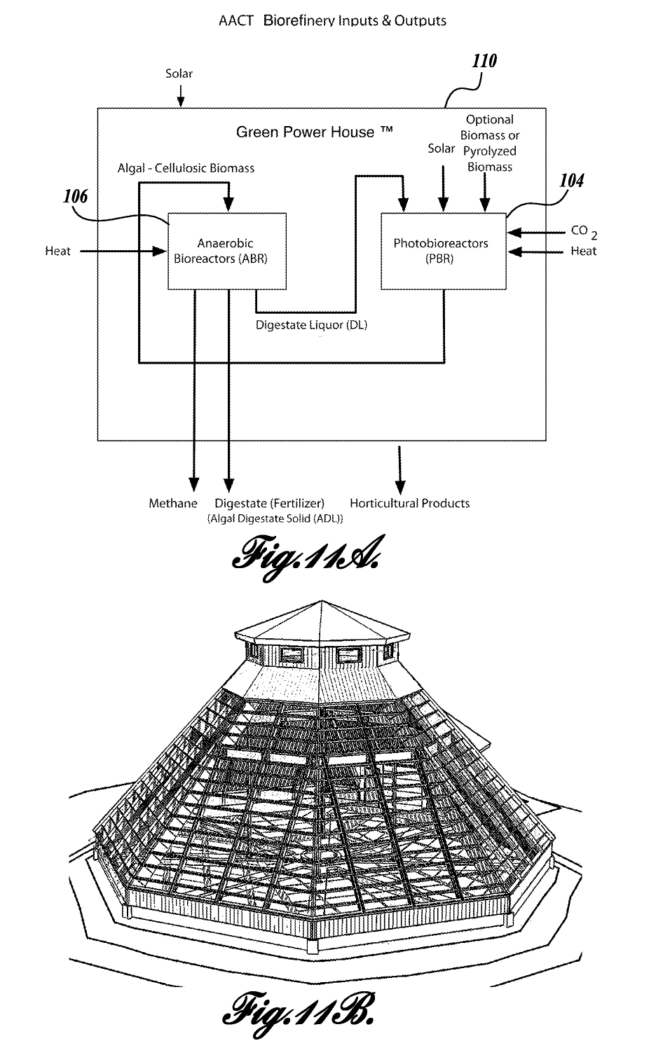

Referring to FIG. 11A, an exemplary schematic of the inputs and outputs of a production scale greenhouse operating on a lumber mill site is shown. The amount of algae that can be produced daily for a 5000 sq. ft, greenhouse biorefinery is approximately 500 gallons of digestate every 5 days. A biomass pyrolysis system can process about 2 to about 12 tons of biomass per day, which produces about 3.5 to about 20 tons of organic carbon every 5 days For a balanced system, the greenhouse biorefinery will produce about 2 tons of organic carbon and about 500 gallons of digestate every 5 days.

The methane and hydrogen can be converted to electrical power, and a large fraction of the digestate can be blended with other waste material at the mill site to produce high value organic soil regenerating products and/or amendments. The combined energy output for a single GPH, producing 2 net tons of organic carbon and 500 gallons of digestate every 5 days is about 250 kWatts produced continuously (about 0.9 MBTU/hr).

Megawatts of continuous power can be obtained by increasing the amount of organic carbon generated daily. The balance of inputs and outputs can be maintained by providing the additional pyrolysis outputs as feedstock for other processes. For example, additional organic carbon can be used in a biofilter reactor, and additional carbon dioxide can be provided to landfills or composting piles to accelerate digestion. Alternatively, a system of multiple biorefineries can be built together to accommodate the additional pyrolysis outputs. The polygonal architecture of the biorefinery makes it easy to create a modular grouping of, for example, six units.

The greenhouse system 110 may use low temperature (<120° F.) thermal and geothermal systems to drive the process. In that regard, heat exchangers and hydronic systems comprising geothermal well water and/or reclaimed process water may be used to keep the algae in the PBRs warm and to keep the anaerobic digestion in the ABRs at the optimal temperatures.

Referring to FIG. 11B, an exemplary greenhouse building is shown. The greenhouse is designed with an octagonal base and having one or more sides configured with windows to receive solar energy.

### Biomass Pyrolysis System

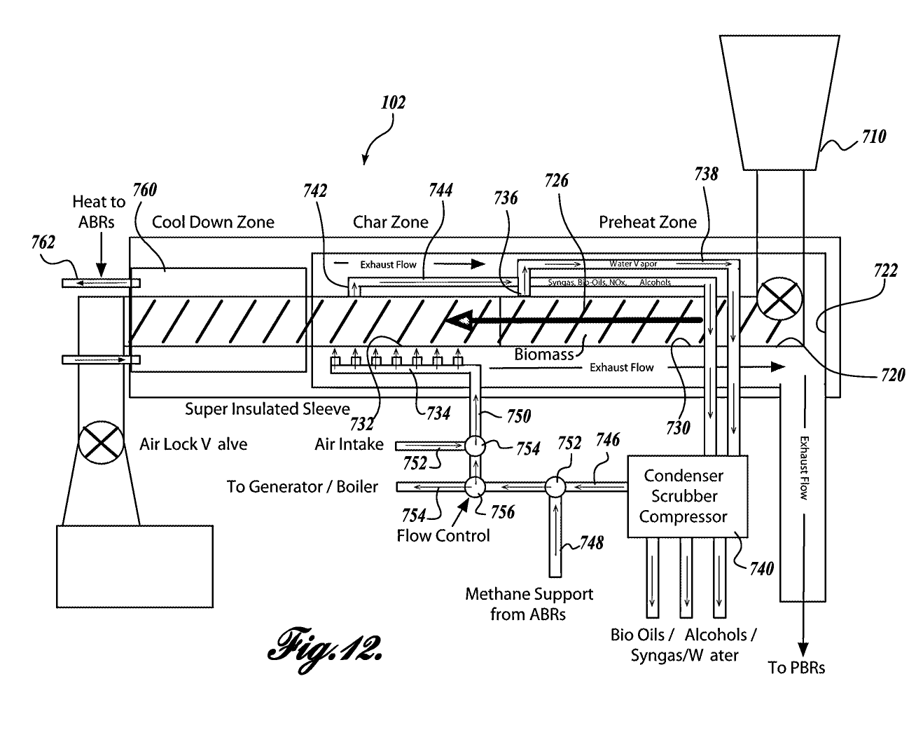

Referring to FIG. 12 a schematic diagram of an exemplary biomass pyrolysis system 102 is shown. Pyrolysis produces a considerable amount of heat and drives off hydrocarbons (for example, in the form of syngas) that can be used as fuel to power the pyrolysis process. Alternatively, or in addition, some of the methane produced by other components in the biorefinery system 100 (for example, the ABR system 106) can be used to start pyrolysis. Once the hydrocarbons begin to flow they are used to power the process.

As can be seen in FIG. 12, the pyrolysis system 102 includes an inlet 710, shown as a feedstock hopper, for receiving biomass. In the illustrated embodiment, the pyrolysis system 102 is a concentric cylindrical system having an inner pyrolysis chamber 720 and an outer exhaust chamber 722 surrounding the inner pyrolysis chamber 720. Between the chambers 720 and 722, the pyrolysis system 102 may include metallic bulkheads to divide the chambers.

When received, the biomass feedstock moves from a feedstock hopper 710 to the pyrolysis chamber 720, for example, using a rotating auger 726. In the pyrolysis chamber 720 biomass is heated to drive off the hydrocarbons, sometimes referred to in the art as “syngas”. Syngas is a gas mixture that includes an intermediate form in the process of making synthetic natural gas (therefore, its nickname “syngas”). Sample syngas components typically include methane, CO (carbon monoxide), carbon dioxide, hydrogen, and sometimes, nitrogen and Noₓ gases (which may be nominal), and can include trace elements of impurities like sulfur.

The pyrolysis chamber 720 may be divided into two zones, a preheat zone 730 and a char zone 732. The preheat zone 730 may be maintained in a temperature range of about 180 F to about 700 F, and preferably in the range of about 200 F to about 600 F. The temperature in the preheat zone 730 may be maintained by a heating device 734 in the char zone 732, as described in greater detail below, or by a separate heating device (not shown).

The primary purpose of the preheat zone 730 is to heat off any water that may be trapped in the feedstock biomass, which boils off at 212 F. The water and other vaporized components are collected at an outlet 736 and travels through line 738 to a system 740 for condensing, scrubbing, and compressing the water and other exhaust from the pyrolysis chamber 720 (for example, but not limited to, syngas, bio-oils, and alcohols, as described below). The water may be reclaimed and used in other systems in a biorefinery system 100, for example, as water in the raceways 402 of the PBR system 104 or as irrigation water for plant life in the greenhouse system 110.

Therefore, the feedstock is dried in the preheat zone 730 in preparation for entry into the char zone 732. In the char zone 732, the preheated biomass feedstock is heated to a temperature in the range of about 600 F to about 1200 F, and more preferably about 700 F to about 850 F. In a non-limiting example, the char zone 732 is configured to heat to about 800 F for about 15 to about 20 minutes. In another embodiment, the microorganisms. Heating may be achieved by a heating device 734, shown as a series of burners, positioned in the char zone 732. The feed gases to the heating device 734 may include methane or hydrogen, for example, from other components in the biorefinery system 100, bio-oils and alcohols collected from the pyrolysis chamber 720, or other combustible gas sources. Exhaust from the heating device 734 is collected in the outer exhaust chamber 722 surrounding the inner pyrolysis chamber 720. The exhaust may include carbon dioxide and other exhaust gases, and flow may be delivered directed to the PBR system 104 as a feedstock for the algal colony.

In the char zone 732, the biomass is converted to biochar or organic carbon. Syngas is collected at an outlet 742 and travels through line 744 to the condenser, scrubber, and compressor system 740. There, bio-oils, alcohols, and water may be condensed, scrubbed, and separated. Any components that may be used to fuel the system heating device 734 may be sent via line 746 to be combined with input methane at line 748 and methane support valve 752 as feed gases to the heating device 734 via line 750. Air intake may also be directed to the heating device 734 via line 752 and air intake valve 754 to combine with line 750. In the alternative, excess gases that are not sent to the heating device 734 may be diverted via flow control valve 756 to a generator or boiler or another system in the biorefinery system 100 via line 754.

After the auger 726 moves the biomass through the preheat and char zones 730 and 732 in the pyrolysis chamber 720, the auger 726 moves the organic carbon to a cool down zone 760, in which one or more heat exchangers 762 collect heat from the biomass. The heat collected by the heat exchangers 762 may be directed to the ABR system 106 (see FIG. 1) or to another system in the overall biorefinery system 100. The cooled organic carbon is then removed from the pyrolysis system 102 as an output.

Depending on the size of the pyrolysis system 102, enough heat can be collected to power both a biorefinery system 100 and a lumber mill, for example, including operating the mill's kiln. Processing 6-30 tons of biomass daily is well within the scope of the system described herein. The system 100 is carbon negative and could also qualify an industrial site utilizing the refinery for further tax rebates and carbon offset trading incentives when carbon legislation passes.

The operation of the biomass pyrolysis system 102 will now be described in greater detail. Initially the system 102 may use either propane or methane delivered to the heating device 734 to start the process. As a non-limiting example, the methane may be an output product from the ABR system 106. Alternatively, an external source such as propane may be used.

When the biomass pyrolysis system 102 produces a sufficient volume of syngas to support the pyrolytic process, the system may be powered by syngas or by a combination of gases. The exhaust gas from the combustion of gases may be vented, cooled, and pumped through the PBR gas bubbler system as feedstock for the algae.

With the heating device 734 on, the char zone 732 comes up to temperature and heats the exhaust chamber 722 surrounding the pyrolysis chamber 720. This in turn heats the preheat zone 730 bringing the biomass feedstock up to temperature, driving off moisture in the form of water vapor as described above. The vapor from the preheat zone 730 may be collected, condensed and distributed to other components in the overall biorefinery system 100, for example, as water feedstock to the PBR system 104.

Excess heat from the pyrolysis chamber 720 may be collected and distributed to other components in the overall biorefinery system 100, as needed, for example, to the PBR and/or ABR systems 104 or 106. Syngas production requires the high temperatures achieved in the char zone 732. The syngas output may be collected and then fractionated, e.g., by means of fractional distillation, and distributed, for example, to the heating device 734 for further powering the pyrolysis system 102. Also, a bubbler or scrubber can be used to separate methane, which does not dissolve in water, from CO₂, which does. The carbon-enriched water then can be transmitted to the PBR system 104 for use as a nutrient input. Excess carbon dioxide not used by the PBR system 104 could be used in alternative way, for example, shunted to feed a compost pile or a landfill waste pile.

As the organic carbon output moves out of the char zone 732, the organic carbon enters a section of the pyrolysis system 102 comprising a heat exchanger 762, such as a water jacket. The heat exchanger process (1) cools the organic carbon such that it reaches ambient temperatures by the time it moves to the output hopper, and (2) collects the excess heat that then can be provided as needed to other member devices, such as the ABR and/or PBR systems 104 and/or 106.

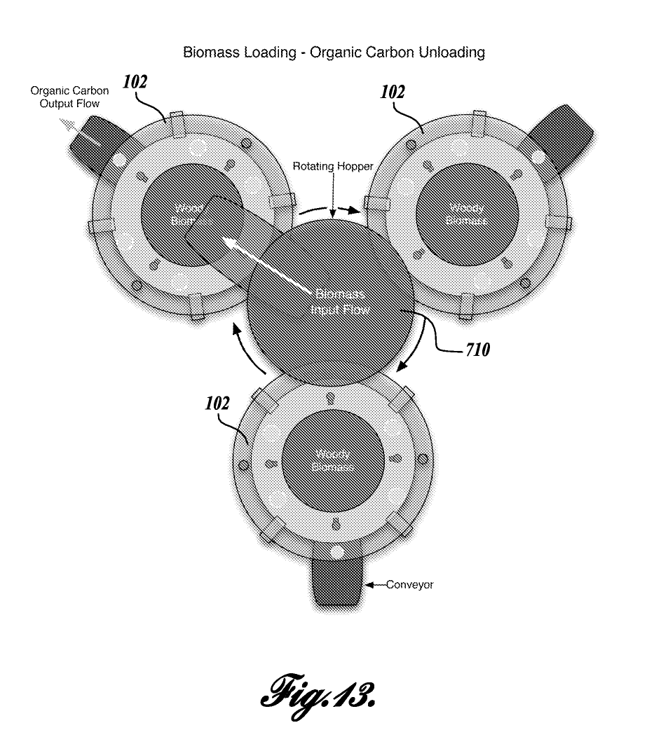

FIG. 13 illustrates one possible configuration for multiple biomass pyrolysis systems 102 sharing a common feedstock hopper. It will be understood by those skilled in the art that other, different configurations are possible. Where an array of biomass pyrolysis systems 102 is utilized, some of the syngas generated by one biomass pyrolysis system 102 can be used to start another biomass pyrolysis system 102. The control system can also direct output gases to the other biomass pyrolysis systems 102, for example, in a round-robin manner, to meet process needs as required.

Preferred organic carbon compositions are generated at temperatures in the range of 800-1000° F., more preferably in the range of 800-900° F. The time it takes to move feedstock through a biomass pyrolysis system 102 will be dependent on a range of variables, including the moisture content of the feedstock, the feedstock species, and the time necessary to remove all syngas, for example, all of which will impact the auger rotation speed. These variables may be managed and controlled by a suitable control system.

In addition, preferred ratios of pyrolysis chamber 720 length to diameter may produce optimal output production. In one embodiment, the preferred length to diameter ratio is 12:1, where pyrolysis chamber 720 length is measured from the start of the preheat zone 730 to the end of the char zone 732 in FIG. 12. In another embodiment, the preferred ratio of preheat zone 730 length to char zone 732 length is 2:1.

A control system may be used in the biomass pyrolysis system 102 to sense and regulate the flow of thermal energy and carbon dioxide through the entire system for the optimal production of biofuels and electricity. Excess heat can be used locally for other industrial processes or diverted into a geothermal storage system for later use, for example, by earth tubes 550 or other geothermal heat exchangers. Organic carbon produced by the biomass pyrolysis system 102 can be blended with the high-nitrogen amendments generated by the ABR system to boost its agricultural and/or soil regenerating value. In addition, the organic carbon output can be used as a substrate for sequestering contaminants, pollutants, and impurities from water supplies, as from a water treatment plant, or waste water from an industrial site, thereby remediating the water and providing a ready collection device for unwanted impurities.

### Example—Biomass Pyrolysis System

Lumber mills typically use their trash wood, known as “hog fuel” (e.g., pulverized bark, shavings, sawdust, low-grade lumber, and lumber rejects) to fuel the kilns that dry their lumber. A medium-size mill that utilizes a standard boiler system for heating its kilns will consume approximately 150 tons of hog fuel a day to fuel its boiler system, which in turn will use between 8,000-25,000 pounds of steam/hour to keeps its kilns at a temperature of 180° F. for a day. A biomass pyrolysis system 102, as described herein, can generate about 2 million BTUs/hr using hog fuel as its feedstock. This quantity of BTUs is capable of generating 30,000 pounds of steam/hour, and would produce approximately 18 tons of quality biochar or organic carbon.

Moreover, adapting a pyrolysis system 102 to such a mill operation allows the mill to take advantage of the pyrolysis system's heat exchange system to support keeping the boiler system's water at temperature. It is calculated that using a pyrolysis system would reduce the boiler system's water temperature fluctuation down to 2 degrees. This reduction alone would reduce the mill's carbon footprint by 60%. Assuming a biomass chamber length to diameter ratio of 12:1 and a preheat zone length to charring zone length ratio of 2:1, an array of 3-5 pyrolysis systems in an overall system configuration would manage a mid-size lumber mill's daily energy needs, as well as the systems energy needs.

### Products

Embodiments of the present disclosure feature systems, components, and methods, for generating a nutrient-dense, organic soil amendment or topsoil substitute or soil regenerating product suitable for organic plant cultivation and other agricultural applications. In one embodiment, an organic soil amendment and/or regenerating products is formed by combining digestate solids and organic carbon in particular ratios to achieve a given, desired consistency and nutrient density. In another embodiment, a soil amendment is formed by combining digestate solids, organic carbon, and digestate liquor in particular ratios to achieve a given, desired consistency and nutrient density. In still another embodiment, a soil amendment is formed by combining digestate solids, organic carbon, digestate liquor, and additional material in particular ratios to achieve a given, desired consistency and nutrient density. The additional material may include, without limitation, soil; waste soil or soil parent material, including pulverized gravel or sand; or clean, non-putrescible landfill, sawdust, hog fuel, or other timber residual biomass.

Below is a range of compositions of components in a suitable soil regenerating product.

In one embodiment of the present disclosure, a soil regeneration product includes a carbon to nitrogen ratio in the range of about 2:1 to about 40:1, and more preferably 4:1 to about 36:1

In another embodiment of the present disclosure, a soil regeneration product includes any of the foregoing or following components and a calcium content in the range of about 0.5 percent to about 6.8 percent, and more preferably about 1.11 to about 6.6 percent.

In another embodiment of the present disclosure, a soil regeneration product includes any of the foregoing or following components and a magnesium content in the range of about 0.25 to about 1.6 percent, and more preferably about 0.33 to about 1.5 percent.

In another embodiment of the present disclosure, a soil regeneration product includes any of the foregoing or following components and a copper content in the range of about 0.73 to about 13 mg/L, and more preferably 1.53 to about 12.03 mg/L.

In another embodiment of the present disclosure, a soil regeneration product includes any of the foregoing or following components and a manganese content in the range of about 100 to about 350 mg/L, and more preferably about 140.2 to about 324.5 mg/L.

In another embodiment of the present disclosure, a soil regeneration product includes any of the foregoing or following components and a nitrogen content in the range of about 0.2 to about 2 percent, and more preferably about 1.1 to about 1.7 percent.

In another embodiment of the present disclosure, a soil regeneration product includes any of the foregoing or following components and a phosphorous content in the range of about 0.4 to about 1.5 percent, and more preferably about 0.9 to about 1.2 percent.

In another embodiment of the present disclosure, a soil regeneration product includes any of the foregoing or following components and a potassium content in the range of about 0.5 to about 7 percent, and more preferably about 0.75 to about 6.5 percent.

In another embodiment of the present disclosure, a soil regeneration product includes any of the foregoing or following components and a sulfate content in the range of about 0.15 to about 1.4 percent, and more preferably about 0.28 to about 1.26 percent.

In another embodiment of the present disclosure, a soil regeneration product includes any of the foregoing or following components and a sodium content in the range of about 0.5 to about 18 percent, and more preferably about 0.14 to about 17.94 percent.

In another embodiment of the present disclosure, a soil regeneration product includes any of the foregoing or following components and a zinc content in the range of about 55 to about 255 mg/L, and more preferably about 84 to about 233.1 mg/L.

In another embodiment of the present disclosure, a soil regeneration product includes any of the foregoing or following components and a iron content in the range of about 600 to about 2500 mg/L, and more preferably about 695.84 to about 2385.92 mg/L.

In another embodiment of the present disclosure, a soil regeneration product includes any of the foregoing or following components and a boron content in the range of about 5 to about 150 mg/L, and more preferably about 6.42 to about 115.7 mg/L.

In another embodiment of the present disclosure, a soil regeneration product includes any of the foregoing or following components and has a pH in the range of about 5.4 to about 9.6.

Embodiments of the present disclosure further may include methods for remediating water by exposing said water to the organic carbon products generated by the systems described herein, and sequestering water contaminants and impurities in the organic carbon. Here organic carbon alone, or in combination with other suitable materials, such as wood chips, fines, or composted material, form a biofilter reactor through which waste water is allowed to flow at a rate sufficient to allow the water's nutrient load to be captured in the porous cells of the organic carbon. In preferred embodiments, the organic carbon comprises at least 10% of the filter, more preferably at least 20%. In another preferred embodiment, organic carbon comprises at least 50%, 70% or 100% of the filtering material in the reactor. In one embodiment the organic carbon biofilter reactor reduces waste water nutrient load by 50%. In another embodiment, it reduces the load by 60%. In still another embodiment, it reduces the load by 70% or more. Another biofilter reactor application, the organic carbon or organic carbon/woodchip combination would filter emissions from flue gas stacks of industrial furnaces.

### Intelligent Control of the Systems

As described above, it has been discovered that intelligent, self-governing, carbon-sequestering devices can be constructed which eliminate undesired biomass waste while producing high value bioenergy outputs or products. These devices can be useful alone or as members of a scalable, extensible, integrated, interactive and cooperative intelligent biorefinery system that mimics the behavior of natural systems.

The management of a biorefinery system 100 and its components, as described herein, requires a sophisticated control system capable of delivering the amount of heat needed for each component or member device of the system, as well as controlling the movement of biomass, gases, heat, and other products through the system. Therefore, each member device is controlled by an autonomous agent, referred to herein as a bioprocessor autonomous agent (or “BPAA”). The autonomous agents are configured to communicate with a governing agent, referred to herein as the biorefinery agent (or “BRA”), which is configured to oversee the entire production process. Adding the autonomous agent component to member devices of the system enables the entire system to be essentially “plug and play.” As more components are added to the biorefinery, the autonomous control system adapts to the added load, redistributing the flow of energy and biomass through the system. Hence, the system is referred to herein as an intelligent biorefinery system, and each member device is itself an intelligent component.

The intelligent member devices of an intelligent biorefinery system are designed to work both in concert with each other and independently. Each component has its own BPAA control system that enables it to adapt to changing environmental conditions and workloads. Multiple intelligent member devices can be interconnected via their BPAAs to form a unique intelligent biorefinery system. In that regard an intelligent biorefinery system can be tailored or adapted for use in numerous industrial or agricultural applications to make these industries and applications cleaner, more efficient, and ultimately more profitable.

For example, where remediation of contaminated water is desired, a member device could be included in an intelligent biorefinery system that is competent to receive both the contaminated water and the organic carbon output from a biomass pyrolysis system as a filter substrate. The member device's BPAA would then control the process of moving the water through the organic carbon at a rate competent to sequester the contaminants in the organic carbon. Purified water and contaminant-laden organic carbon would be outputs of the device and could be accessible to other member devices via the system, as appropriate. This member device could be designed and built specifically for the system, or an existing device could be adapted to plug into the intelligent biorefinery system simply by modifying the device so that it is competent to receive the new component. In one embodiment, the device is modified by means of an adapter that communicates between the device and the BPAA.

Another example of tailoring an intelligent biorefinery system for a given industry to improve its function is in the waste management or water treatment industries. One issue for these industries is that standard anaerobic digestion of the organic sludge or slurry does not breakdown any pharmaceuticals or hormones that may accumulate in the waste sludge. This requires heating the material to at least 600° F. Thus, a tailored intelligent biorefinery system could receive the sludge or slurry, de-water it as necessary, and add it as feedstock to an ABR to digest or breakdown the organic material. The ABR digestate output then could be dried as needed and provided as feedstock to a biomass pyrolysis device having heating capabilities sufficient to breakdown the hormones and pharmaceuticals remaining in the sludge digestate. The biomass pyrolysis output then could be returned to the earth for horticultural applications or forest remediation, as examples. Alternatively, if the treatment plant provides its own means for digesting it waste, the ABR step could be eliminated.

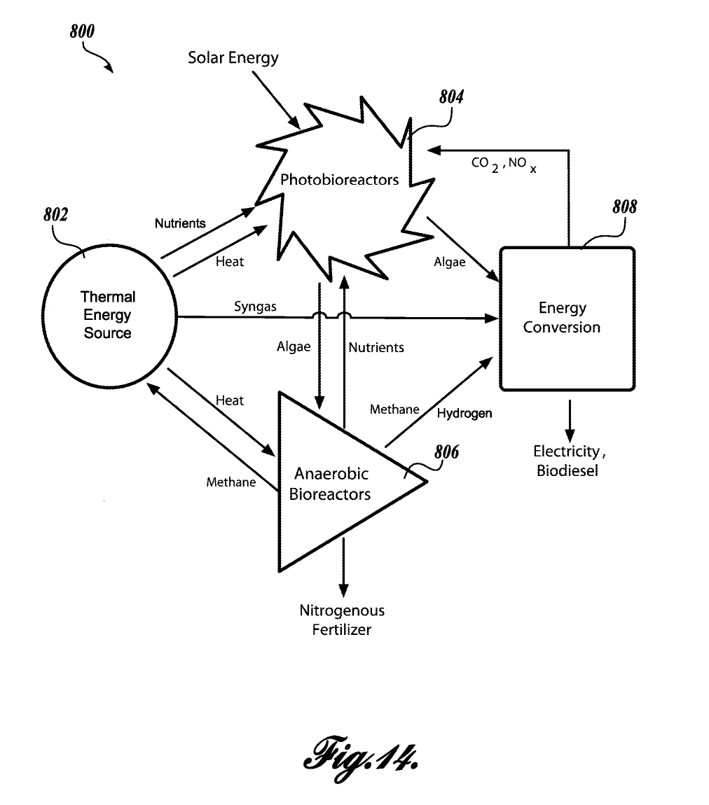

The intelligent biorefinery systems described herein are designed to integrate with existing industries that generate waste heat and carbon dioxide, providing a system for sequestering carbon, reclaiming the waste heat, and generating bioenergy products of value. Referring to FIG. 14 an exemplary intelligent biorefinery system is shown. Similar to FIG. 1, this schematic illustrates the four basic components that make up an intelligent biorefinery system 800—thermal energy source 802, photobioreactor 804, anaerobic bioreactor 806, and energy conversion 808. This schematic also illustrates the opportunity for sharing inputs and outputs cooperatively among the member devices in a manner that supports the optimal production of the overall system. The BPAAs are designed to control the member devices to support optimal productions of the overall system.

The BPAAs give each member device the means for solving complex nonlinear problems that can arise while attempting to maintain a stable biological environment in changing conditions. The control system also assists in the harvesting and processing of the algal biomass to produce biofuels, electricity and nitrogenous fertilizer and soil regenerating products.

Each member device of an intelligent biorefinery system, in accordance with embodiments of the present disclosure produces a bioenergy product using a process based on simple biological principals. The intelligent biorefinery system takes this concept to the next level through the use of adaptive behavioral controls that mimic natural biological processes.

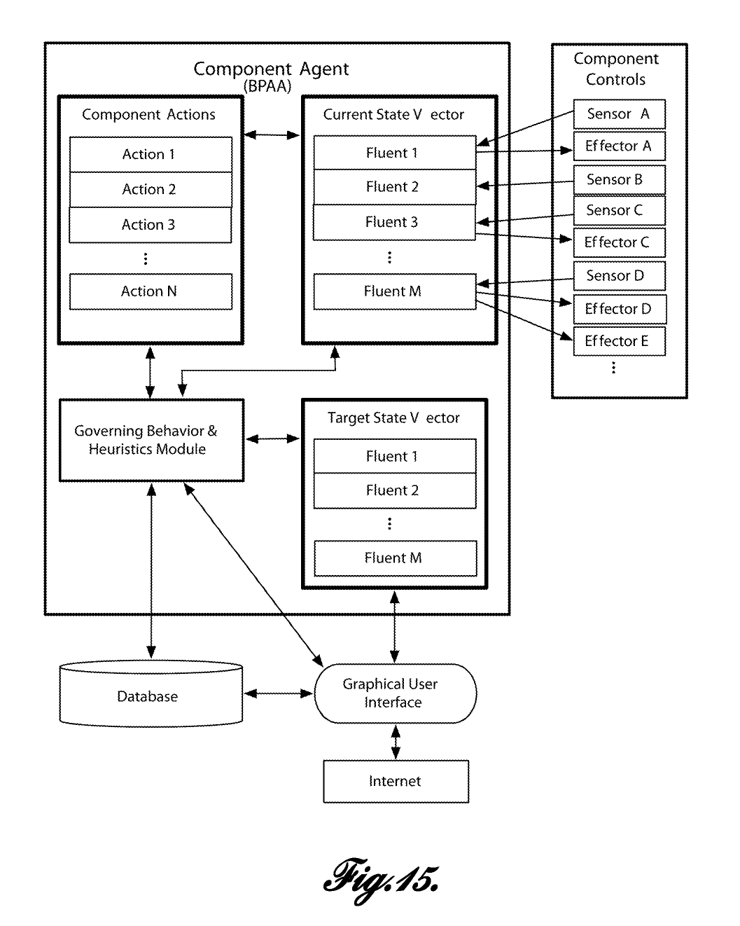

The component autonomous agents or BPAAs will now be described. As mentioned above, the functionality of each component or member device of an intelligent biorefinery system is governed by an autonomous agent, such as a software agent, referred to herein as a BPAA. As illustrated in the flow chart in FIG. 15, an agent comprises four basic subcomponents:
A Current State Vector that functionally describes the current state of the component.
A Target State Vector that describes the desired state of the component.
A set of Actions the component can perform to modify its current state.
A Behavioral Module that determines what actions the component needs execute to achieve or maintain the Target State.

FIG. 15 illustrates how information flows between the agent's subcomponents as well as the flow of data between the physical sensors and control mechanisms (effectors) that modify the physical state of the component.

The Current State Vector and the Target State Vector are composed of software objects known as Fluents. Fluents are variables that can be single valued, represent a range of values, or can be connected to a sensor to represent a measured physical parameter. For example, the Current State of a BPAA can have a fluent called “Raceway Temperature,” with a sensed value of 80° F., while the Target State can have a fluent called “Raceway Temperature” that has an interval value between 78 and 82° F., written as [78-82]. The BPAA behavior module recognizes that 80° is within the range [78-82] and so does not need to perform any actions to modify the temperature of the photobioreactor raceway. Fluents also could include Interval Valued Fluents. An example is a goal state temperature Fluent that is set for the interval range [75, 90] degrees and a current state temperature Fluent that is “sensed” at 80° F. In this case, the temperature component of the state vector would be a match.

Component behaviors can be reactive, predictive, or adaptive, or a combination of these. A reactive behavior constantly executes actions to adjust the current state to match the target state, such as opening or closing a heat exchanger valve to adjust the temperature in a component so that it matches the target state temperature. A predictive behavior might use information such as a weather forecast gathered from the Internet to begin adjusting the temperature in anticipation of a sudden cold snap. An adaptive behavior can combine predictive and reactive behaviors to generate new behaviors based on the best outcome.

The entire intelligent biorefinery system may also have its own BPAA, which has a similar structure to the member device BPAAs of the system, but is designed to oversee the system and each of the component agents. As mentioned above, such an agent is referred to herein as a governing agent or Biorefinery Agent (BRA). In this case each component agent is considered a fluent of the BRA.

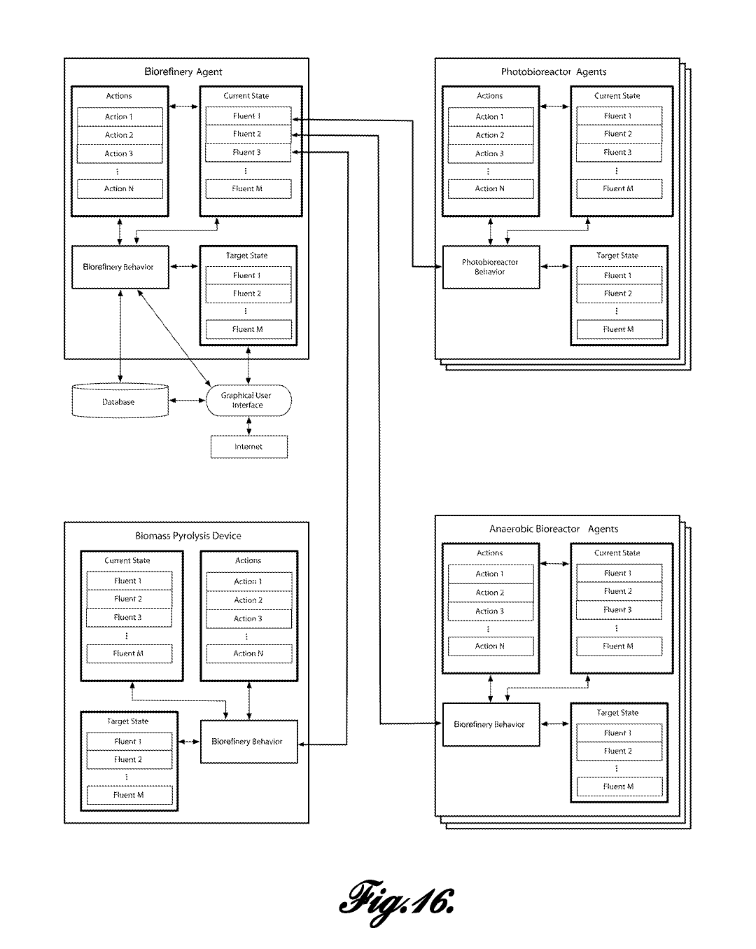

FIG. 16 shows the control strategy for an intelligent biorefinery system that has multiple photobioreactors and anaerobic bioreactors and an agent that controls a geothermal heat source for the system. Each autonomous agent is responsible for maintaining the “state” of a single component and controlling the flow of material on these busses (biomass, CO₂, heat, etc.). The behavior module of each component BPAA and the BRA can be thought of as non-linear systems solver that uses actions to modify the state of a component or member device. The BPAA compares the current state of the member device to the target state (the Goal) to what actions need to be taken.

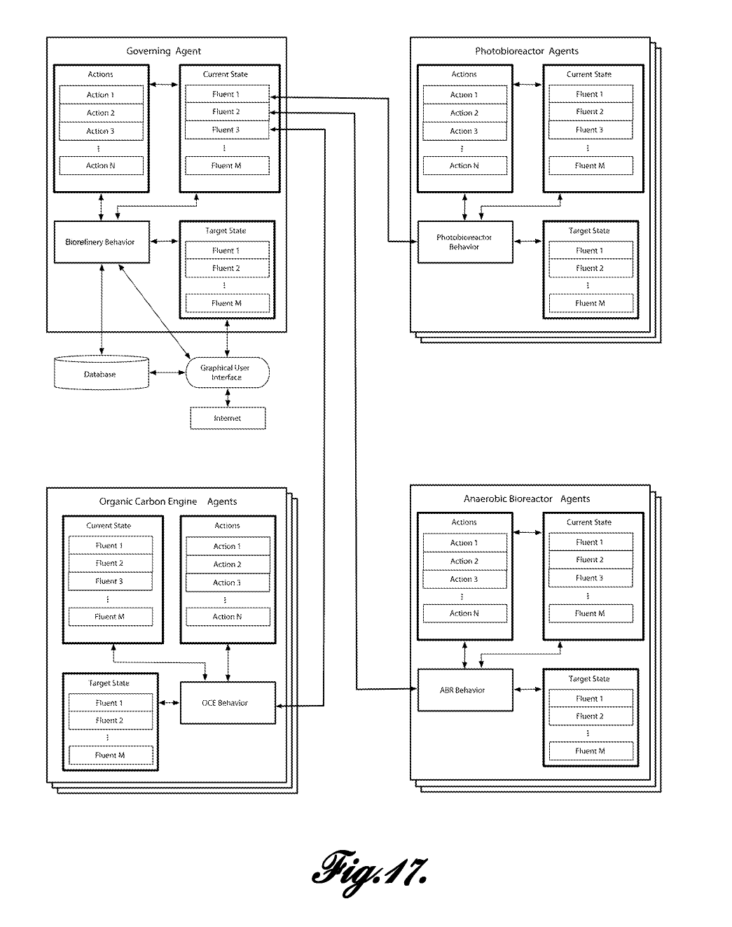

FIGS. 1 and 14 are schematic diagrams of intelligent biorefinery systems in accordance with embodiments of the present disclosure. These FIGURES illustrate the inputs and outputs of each member device and how various outputs can be shared as inputs across the system. For example, FIG. 14 depicts an intelligent biorefinery system utilizing a generic thermal heat source as a member device, and FIG. 1 depicts an intelligent biorefinery system wherein the heat source member device is a biomass pyrolysis system. FIG. 17 may be a flow chart for the intelligent biorefinery system depicted in FIG. 14, depicting the communication pathways among the member devices that allow the inputs and outputs to be shared across the system as depicted in FIG. 14. Similarly, FIG. 16 may be a flow chart for the intelligent biorefinery system depicted in FIG. 1, depicting the communication pathways among the member devices that allow the inputs and outputs to be shared across the system as depicted in FIG. 1.

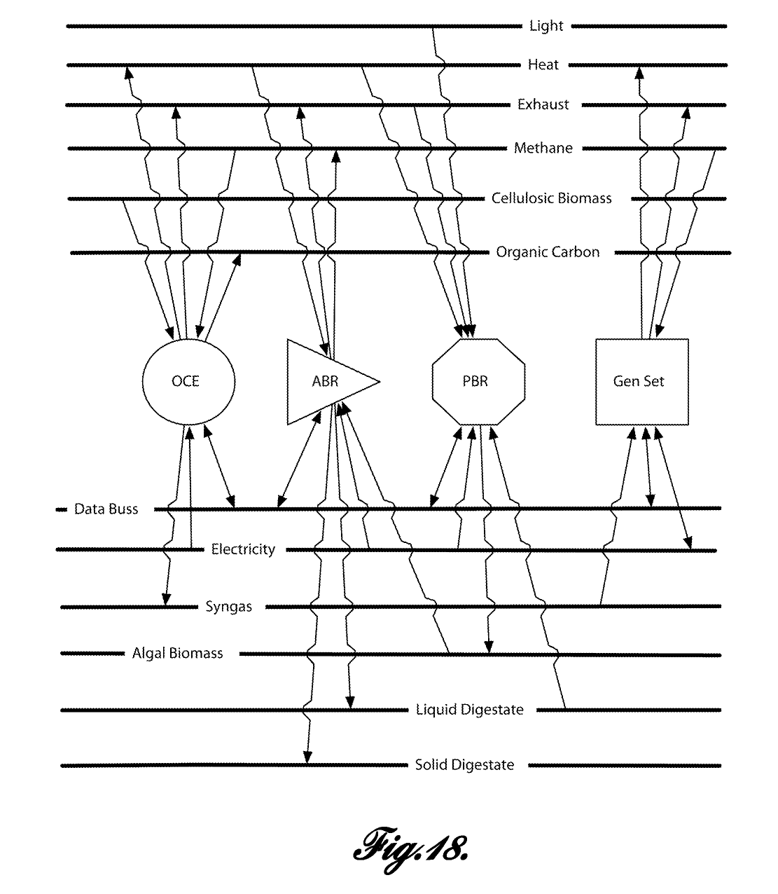

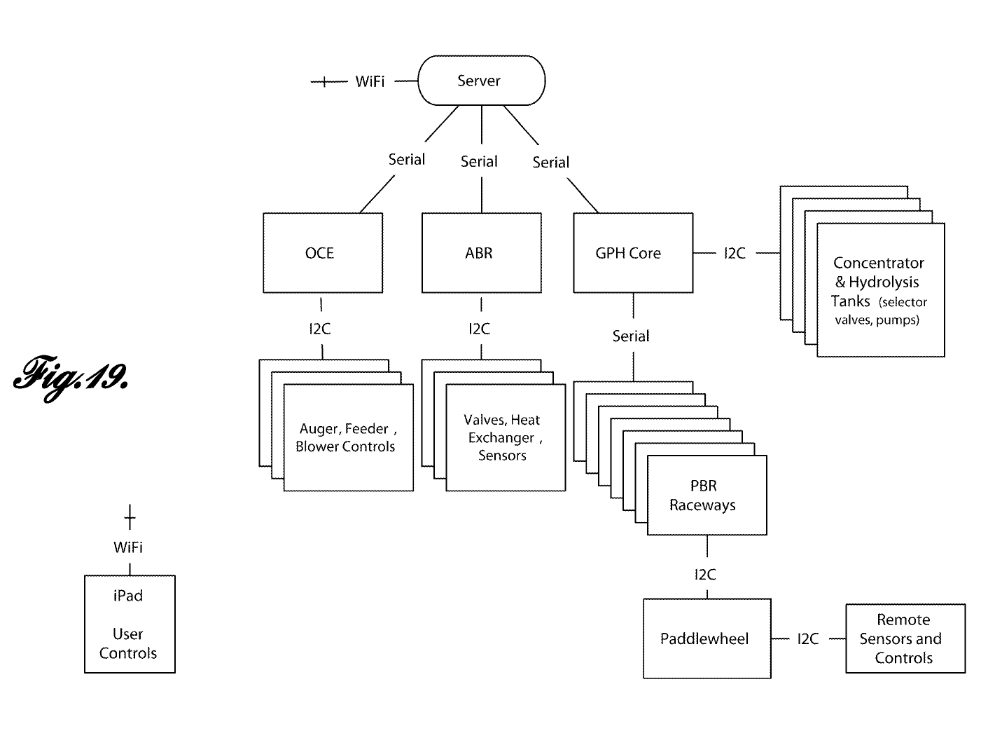

FIG. 18 is another flow chart depicting both the inputs and outputs of an intelligent biorefinery system as described in FIGS. 1 and 13, as well as the communication means for sharing information, as depicted in FIGS. 16 and 17. In FIG. 18, all member device behavior information is communicated to the BRA BPAA and received from the BRA BPAA by means of the data buss “line” in the drawing. This is indicated in the drawing by means of a bi-directional arrow between member devices and the Data Buss line. Member device inputs and outputs and how they are shared across the system is indicated by appropriately marked arrows leading to and from reference lines in the drawing representing, for example, methane, algal biomass, or organic carbon.

Looking at the biomass pyrolysis system 102 schematic of FIG. 12 as an exemplary member device, let us say the system wants to start the biomass pyrolysis system up in the morning. This information is communicated to the biomass pyrolysis system from the BRA (FIG. 16), via the data buss line in FIG. 18. The BPAA of the biomass pyrolysis system 102 evaluates its current state via the fluents in the current state vector, and begins to initiate appropriate actions, given the desired target state communicated from the BRA (see FIG. 16).

Target state vector information might include being on for a certain amount of time, producing a desired amount of organic carbon, utilizing a preferred feedstock, and/or generating a desired amount of heat, syngas or methane (see FIGS. 1 and 14). Based on the data perceived as the biomass pyrolysis system's current state, the biomass pyrolysis system's BPAA behavior module will initiate a series of Actions, communicated to Effectors via the Fluents (FIG. 15).

Exemplary actions may include opening the methane support valve 752 to receive methane from intelligent biorefinery systems (see FIG. 12 and FIG. 18). This behavior is communicated via the buss line to the BRA and the member device intelligent biorefinery system whose BPAA governing behavior module now knows its behavior has changed and that methane support is needed by another member device. The intelligent biorefinery system BPAA then initiates a series of Actions (e.g., release methane, collect methane, or increase digestate production, depending on the current state of the ABR device as perceived by its governing behavior module, see FIG. 16), ultimately providing methane to the biomass pyrolysis device 102 by means of the representative methane line 748 in FIG. 18. As will be understood by those skilled in the art and described herein above, the system is designed for continual device analysis, as well as predictive, reactive, and/or adaptive behaviors, allowing the system to function optimally, cooperatively and harmoniously in a continually adapting manner.

The intelligent biorefinery system design also allows a given intelligent biorefinery system to communicate with other intelligent biorefinery systems that may be local or at a distance by means of its governing behavior module, and to share that information with its member device BPAAs. For example, an intelligent biorefinery system located in Montana might be experiencing climate conditions commonly experienced in Hawaii, and which might particularly impact algal growth in the Montana intelligent biorefinery system. Using the system described herein, the Montana intelligent biorefinery system can access the Hawaii intelligent biorefinery system behavior information, and the Montana intelligent biorefinery system BPAA can utilize that solution information as part of its solution path for initiating action(s) intended to move the intelligent biorefinery systems behavior to its desired target state. Clearly, as will be understood by those skilled in the art, the Montana intelligent biorefinery system also is competent to share its behavior information with the Hawaii intelligent biorefinery system or other intelligent biorefinery systems.

This ability to communicate across systems has particular application in the embodiment where multiple intelligent biorefinery systems work together at a local industrial application. For example, one embodiment of the disclosure is an array of two or more intelligent biorefinery systems, wherein the BRA is an intelligent green house. In another embodiment the green house is octagonal in shape and multiple greenhouses may be arrayed in a honeycomb pattern, allowing them all to share resources, including thermally stored heat on their common side.

The BPAA intelligent process controls described herein allow one to tailor the design of an intelligent biorefinery system to a target industry with minimal programming, using a standard set of components. It also allows one to modify an existing non-intelligent device so that it can participate as an intelligent biorefinery system member device. In this case, the additional step required would be adapting, as necessary, the physical sensor and effector mechanisms so they are competent to receive information from, and effect changes on, the device.

Adaptation can be accomplished by using an adapter means that interface with the BPAA and the device to be modified. Thus, the adapter means can be modified as needed to work with a wide range of currently existing devices allowing them to participate in an intelligent biorefinery system, without needing to substantially modify the intelligent biorefinery system itself or to re-design or build whole devices anew. Thus, a “plug in-and-play” intelligent, carbon-sequestering intelligent biorefinery system now is available for use in multiple different industries. In the lumber mill example described above, if one wanted to include the mill's boiler as part of an intelligent biorefinery system, such an adapter means might include sensors for measuring water temperature, and effectors for modulating the quantity of heat provided to the boiler.

In accordance with aspects of the present disclosure, the systems described herein may be intelligent biorefinery systems. Intelligent biorefinery systems are interactive systems including integrated, cooperatively-acting member devices and which may use artificial intelligence to (1) govern the behavior of each member device autonomously, and (2) communicate that behavior to one or more other member devices through an autonomous agent that acts as a governing agent. In that regarding the behaviors of the member devices and the system itself are designed such that the member devices function cooperatively, modulating their individual inputs and outputs based on the needs of the system.

In accordance with aspects of the present disclosure, each member device is itself an autonomous agent, which may be competent to (1) perceive the current state of the member device, using sensors and effectors, respectively, to perceive and act on its environment; (2) identify a target state based on input from its local environment and other resources including, without limitation, databases, other systems or devices in other locations, and/or a governing agent; (3) initiate action(s) intended to modify the member device's behavior towards the desired target state; and (4) evaluate the success or failure of initiated actions in achieving the target state, and make changes accordingly.

In accordance with aspects of the present disclosure, the autonomous agent includes in its solution process the outcomes of previous solution pathways sought, effectively continually “learning”. In another aspect, the autonomous agent mimics nature's own process for continually evolving and adapting to changes in the environment, dynamically balancing inputs and outputs while discovering the “best” process for achieving a desired result. In other aspects, the autonomous agent utilizes a goal-directed behavior model as part of its solution process. In another aspect, the autonomous agent utilizes a heuristic algorithm or function as part of its solution path. In still another aspect, the autonomous agent utilizes fluents as part of the process of understanding its current and target states, and/or as a means for (1) communicating computed actions to effectors in the external environment, and (2) communicating the state of the external environment to the autonomous agent perceived through one or more sensors.

In accordance with aspects of the present disclosure, the autonomous agents of the intelligent biorefinery system member devices may have a common architecture and structure, allowing the member devices to easily plug into or out of the system as needed, enhancing the portability and extendability of the intelligent biorefinery system, as well as its modification for multiple, different industries or applications.

In accordance with aspects of the present disclosure, the PBR autonomous agent acts as a system's Governing Agent. In still another aspect, the facility or structure that houses the member devices (e.g., the greenhouse system) may act as a Governing Agent. In another aspect, the greenhouse system has value as a functional greenhouse.

In another aspect, the embodiments of the disclosure feature intelligent components, each of which includes an autonomous agent as described herein.

Embodiments of the disclosure may be embodied in other specific forms without departing from the spirit or essential characteristics thereof. The present embodiments are therefore to be considered in all respects as illustrative and not restrictive, the scope of the disclosure being indicated by the appended claims rather than by the foregoing description, and all changes that come within the meaning and range of equivalency of the claims are therefore intended to be embraced therein. While illustrative embodiments have been illustrated and described, it will be appreciated that various changes can be made therein without departing from the spirit and scope of the disclosure.

1. A biorefinery system, comprising:
(a) a photobioreactor system;
(b) an anaerobic bioreactor system; and
(c) an enclosure for containing at least a portion of the photobioreactor system and at least a portion of the anaerobic bioreactor system, wherein the enclosure has an environment for growing plant life.

2. The biorefinery system of claim 1, wherein the photobioreactor system is configured to grow an algal colony and produce an algal harvest.

3. The biorefinery system of claim 2, wherein the anaerobic bioreactor system is configured to consume the algal harvest to produce one or more products selected from the group consisting of methane, carbon dioxide, hydrogen, and fertilizer.

4. The biorefinery system of claim 1, wherein a feedstock to the photobioreactor system is an exhaust gas from an external system.

5. The biorefinery system of claim 4, wherein the external system is selected from the group consisting of a biomass pyrolysis system, an energy conversion system, an anaerobic bioreactor, and a flue gas stack.

6. The biorefinery system of claim 1, wherein a feedstock to the photobioreactor system is at least a portion of the fertilizer output from the anaerobic bioreactor system.

7. The biorefinery system of claim 1, wherein plant life irrigation water is received from reclaimed water from a biomass pyrolysis system.

8. The biorefinery system of claim 1, wherein the photobioreactor receives reclaimed water from a biomass pyrolysis system.

9. The biorefinery system of claim 1, wherein the enclosure is designed to receive solar energy.

10. The biorefinery system of claim 1, wherein the enclosure is designed to receive heat from at least one of an external system, a hydronics system, and geothermal heat.

11. The biorefinery system of claim 1, wherein the system has managed inputs and outputs.

12. The biorefinery system of claim 1, further comprising a control system including a plurality of autonomous agents for controlling a plurality of components in the system, wherein one of the plurality of autonomous agents is a governing agent.

13. The biorefinery system of claim 12, wherein the control system may be reactive, predictive, adaptive, or a combination thereof.

14. The biorefinery system of claim 12, wherein the control system may be adaptive by using a solution process selected from the group consisting of goal-directed behavior models, heuristic algorithms, and fluents.

15. The biorefinery system of claim 14, wherein the solution process is competent to adapt to changes in its environment.

16. The biorefinery system of claim 12, wherein the control system may receive information from another biorefinery system.

17. A method of growing plant life in a greenhouse system, the method including:
(a) forming an enclosure, wherein at least a portion of the enclosure is configured for receiving solar energy;
(b) disposing at least a portion of a photobioreactor system in the enclosure; and
(c) disposing at least a portion of an anaerobic bioreactor system in the enclosure.

18. The method of claim 17, further comprising growing an algal colony in the photobioreactor system to produce an algal harvest.

19. The method of claim 18, further comprising consuming the algal harvest in the anaerobic bioreactor system to produce one or more products selected from the group consisting of methane, hydrogen, and fertilizer.

20-24. (canceled)

25. A photobioreactor system for growing an algal colony, the system comprising:
(a) a source of exhaust gas;
(b) a raceway system including a plurality of raceways configured to consume the exhaust gas to grow an algal colony; and
(c) a valve system for draining the algal colony from at least one of the raceways, wherein each of the plurality of raceways is positioned to be adjacent the valve system.

26-62. (canceled)
